SEQUENCE LISTING (45 sequences)

SEQ ID NO: 1 - protein, 274 aa, Staphylococcus aureus
  misc_feature (1)..(274): Wild-Type PurR Protein Sequence
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKGVSVLVESKEVKQRLIEDYTSLVKLSDVDEYNQEFNVEPGNSLSKFS

SEQ ID NO: 2 - protein, 274 aa, Artificial Sequence
  Synthetic PurR Q52P
MRYKRSERIVFMTQYLMNHPNKLIPLTFFVKKFKQAKSSISEDVQIIKNTFPKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKGVSVLVESKEVKQRLIEDYTSLVKLSDVDEYNQEFNVEPGNSLSKFS

SEQ ID NO: 3 - protein, 228 aa, Artificial Sequence
  Synthetic Deletion affecting V229  frame shift
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKG

SEQ ID NO: 4 - protein, 70 aa, Artificial Sequence
  Synthetic Y71stop
MRYKRSERIVFMTQYLMNHPNKLIPLTFFVKKFKQAKSSISEDVQIIKNTFQKEKLGTVITTAGASGGVT

SEQ ID NO: 5 - protein, 274 aa, Artificial Sequence
  Synthetic V156E
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPEVVIRKDNKVTEGSTVSINYVSGSSRKIETMVLSKRTLAENSNVLVVDDFMRAGGSINGVMNLMNEFKAHVKGVSVLVESKEVKQRLIEDYTSLVKLSDVDEYNQEFNVEPGNSLSKFS

SEQ ID NO: 6 - protein, 171 aa, Artificial Sequence
  Synthetic S172stop
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTV

SEQ ID NO: 7 - protein, 274 aa, Artificial Sequence
  Synthetic H225D
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKADVKGVSVLVESKEVKQRLIEDYTSLVKLSDVDEYNQEFNVEPGNSLSKFS

SEQ ID NO: 8 - protein, 229 aa, Artificial Sequence
  Synthetic S230top
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKGV

SEQ ID NO: 9 - protein, 239 aa, Artificial Sequence
  Synthetic Q240stop
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKGVSVLVESKEVK

SEQ ID NO: 10 - protein, 274 aa, Artificial Sequence
  Synthetic R96A
MRYKRSERIVFMTQYLMNHPNKLIPLTFFVKKFKQAKSSISEDVQIIKNTFQKEKLGTVITTAGASGGVTYKPMMSKEEATEVVNEVITLLEEKEALLPGGYLFLSDLVGNPSLLNKVGKLIASIYMEEKLDAVVTIATKGISLANAVANILNLPVVVIRKDNKVTEGSTVSINYVSGSSRKIETMVLSKRTLAENSNVLVVDDFMRAGGSINGVMNLMNEFKAHVKGVSVLVESKEVKQRLIEDYTSLVKLSDVDEYNQEFNVEPGNSLSKFS

SEQ ID NO: 11 - protein, 274 aa, Artificial Sequence
  Synthetic V148F
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAFANILNLPVVVIRKDNKVTEGSTVSINYVSGSSRKIETMVLSKRTLAENSNVLVVDDFMRAGGSINGVMNLMNEFKAHVKGVSVLVESKEVKQRLIEDYTSLVKLSDVDEYNQEFNVEPGNSLSKFS

SEQ ID NO: 12 - protein, 44 aa, Artificial Sequence
  Synthetic Q45stop
MRYKRSERIVFMTQYLMNHPNKLIPLTFFVKKFKQAKSSISEDV

SEQ ID NO: 13 - protein, 13 aa, Artificial Sequence
  Synthetic Q14stop
MRYKRSERIVFMT

SEQ ID NO: 14 - protein, 90 aa, Artificial Sequence
  Synthetic Insertion affecting L91 -->frame shift
MRYKRSERIVFMTQYLMNHPNKLIPLTFFVKKFKQAKSSISEDVQIIKNTFQKEKLGTVITTAGASGGVTYKPMMSKEEATEVVNEVITL

SEQ ID NO: 15 - protein, 195 aa, Artificial Sequence
  Synthetic Deletion affecting N196 --> frame shift
MRYKRSERIVFMTQYLMNHPNKLIPLTFFVKKFKQAKSSISEDVQIIKNTFQKEKLGTVITTAGASGGVTYKPMMSKEEATEVVNEVITLLEEKERLLPGGYLFLSDLVGNPSLLNKVGKLIASIYMEEKLDAVVTIATKGISLANAVANILNLPVVVIRKDNKVTEGSTVSINYVSGSSRKIETMVLSKRTLAE

SEQ ID NO: 16 - DNA, 825 bp, Staphylococcus aureus
  misc_feature (1)..(825): Wild-Type PurR Nucleotide Sequence
atgagatataaacgaagcgagagaattgtttttatgacgcaatatttgatgaaccatccgaataaattgattccattaactttttttgtgaaaaaatttaaacaggcgaagtcttcaataagtgaagatgtccaaattataaaaaatacattccaaaaagaaaagttaggtacagtaattactactgctggcgcaagtggtggtgttacgtataaaccaatgatgagtaaagaagaggcgactgaagttgttaatgaggtcattactctattagaagagaaagaacgtttgttacctggcggatatttatttttatcagatttggtaggtaatccatcgctactaaacaaagttggtaagttaattgccagtatttacatggaagaaaaattagatgctgttgttaccattgcgacaaaaggtatttcattggcaaatgcggttgctaatattttaaatttaccagtagtagtgattagaaaagacaacaaggtgactgaaggttctacagtttcaattaattacgtttcaggatcttcaagaaaaatagaaacaatggtactttcgaagagaactttagcagaaaattcaaatgttttagttgtcgatgattttatgagggctggtggctctattaatggtgttatgaatttaatgaatgagtttaaagcccatgtaaaaggggtatcagtacttgtagaatcaaaagaagttaaacaaagattgattgaagattatacttccttagtgaaattatctgatgtagatgaatataatcaagagtttaacgtagaacctggcaacagtttatctaagttttcataa

SEQ ID NO: 17 - DNA, 34 bp, Artificial Sequence
  Synthetic PurR F
tttggtaccatatcttgaaaagtggtgcagatgg

SEQ ID NO: 18 - DNA, 34 bp, Artificial Sequence
  Synthetic PurR R
tttgagctccctgcttcttccaaaacaaccttta

SEQ ID NO: 19 - DNA, 21 bp, Artificial Sequence
  Synthetic pALC MCS F
ataccgcacagatgcgtaagg

SEQ ID NO: 20 - DNA, 25 bp, Artificial Sequence
  Synthetic pALC MCS R
cgatgacttagtaaagcacatctaa

SEQ ID NO: 21 - DNA, 56 bp, Artificial Sequence
  Synthetic FnbAB Up F
ggggacaagtttgtacaaaaaagcaggctcacagatacttccaagattctcaaacc

SEQ ID NO: 22 - DNA, 39 bp, Artificial Sequence
  Synthetic FnbAB Up R
ggacctccgcggcagtggaacaaggtaaagtagtaacac

SEQ ID NO: 23 - DNA, 39 bp, Artificial Sequence
  Synthetic FnbABDown F
ggacctccgcgggtattcaagtcatcagaaacccttgtc

SEQ ID NO: 24 - DNA, 56 bp, Artificial Sequence
  Synthetic FnbABDown R
ggggaccactttgtacaagaaagctgggtcagggcctatatttaacaaagttgcac

SEQ ID NO: 25 - DNA, 32 bp, Artificial Sequence
  Synthetic pGYluxFnbA F
gcgcccggggcaatatattgccttgaaacacg

SEQ ID NO: 26 - DNA, 31 bp, Artificial Sequence
  Synthetic pGYluxFnbA R
gcggtcgactataatatctccctttaaatgc

SEQ ID NO: 27 - DNA, 32 bp, Artificial Sequence
  Synthetic pGYluxFnbB F
gcgcccggggtgttttctgattgcttcattgc

SEQ ID NO: 28 - DNA, 31 bp, Artificial Sequence
  Synthetic pGYluxFnbB R
gcggtcgactataatattctcccttaaatgc

SEQ ID NO: 29 - DNA, 29 bp, Artificial Sequence
  Synthetic PurR His F
tttggatccggtccaagtgcttccggtaa

SEQ ID NO: 30 - DNA, 30 bp, Artificial Sequence
  Synthetic PurR His R
tttcatatgagatataaacgaagcgagaga

SEQ ID NO: 31 - DNA, 20 bp, Artificial Sequence
  Synthetic PurE F
gggcagttcttccgattgga

SEQ ID NO: 32 - DNA, 20 bp, Artificial Sequence
  Synthetic PurE R
ctgttcgcccttgactgcta

SEQ ID NO: 33 - DNA, 29 bp, Artificial Sequence
  Synthetic FnbA F
tttggatcctgtgcgtattgtacaggcga

SEQ ID NO: 34 - DNA, 29 bp, Artificial Sequence
  Synthetic FnbA R
tttgagctcagccgtatttcaagccgaca

SEQ ID NO: 35 - DNA, 27 bp, Artificial Sequence
  Synthetic qPCR PurE F
cttctgaagcgagagaaagaggtataa

SEQ ID NO: 36 - DNA, 27 bp, Artificial Sequence
  Synthetic qPCR PurE R
caataactggtagcgtcgttaatgatg

SEQ ID NO: 37 - DNA, 24 bp, Artificial Sequence
  Synthetic qPCR FnbA F
cggcattagaaaacataaattggg

SEQ ID NO: 38 - DNA, 26 bp, Artificial Sequence
  Synthetic qPCR FnbA R
gttttattatcagtagctgaattccc

SEQ ID NO: 39 - DNA, 20 bp, Artificial Sequence
  Synthetic qPCR FnbB F
gaaaacacaaattgggagcg

SEQ ID NO: 40 - DNA, 21 bp, Artificial Sequence
  Synthetic qPCR FnbB R
tgtttcgcttgctttactttc

SEQ ID NO: 41 - DNA, 14 bp, Bacillus subtilis
wwwhvcgaayrwtw

SEQ ID NO: 42 - DNA, 13 bp, Lactococcus lactis
awwwccgaacwwt

SEQ ID NO: 43 - DNA, 70 bp, Artificial Sequence
  Synthetic purE
tcaaaataaagttcgatttttgattgaaaaagcagaaattgcttgttatgctatatctataatatacaac

SEQ ID NO: 44 - DNA, 70 bp, Artificial Sequence
  Synthetic purA
aaaacgatttgttaaaatgatttttcttttaaaaaggccgaaaatcaatgttcgatttttatttgcatta

SEQ ID NO: 45 - DNA, 70 bp, Artificial Sequence
  Synthetic fnbA
aaaattaatgacaatcttaacttttcattaactcgcttttttgtattgcttttaaaaaccgaacaatata

(19) United States
(12) Patent Application Publication
(10) Pub. No.: US 2022/0195018 A1
(43) Pub. Date: Jun. 23, 2022

(54) ATTENUATION OF BACTERIAL INFECTION

(71) Applicant: THE UNIVERSITY OF WESTERN ONTARIO, London (CA)

(72) Inventors: David E. HEINRICHS, London (CA); Ronald S. FLANNAGAN, London (CA)

(73) Assignee: THE UNIVERSITY OF WESTERN ONTARIO, London, ON (CA)

(21) Appl. No.: 17/429,051

(22) PCT Filed: Feb. 5, 2020
(86) PCT No.: PCT/CA2020/050142

§ 371 Date: Aug. 6, 2021

Related U.S. Application Data
(60) Provisional application No. 62/801,958, filed on Feb. 6, 2019.
(60) Provisional application No. 62/805,295, filed on Feb. 13, 2019.

(51) Int. Cl. C07K 16/12; A61P 31/04; C12N 15/11; C12N 9/22; C12N 15/74

(52) U.S. Cl. CPC C07K 16/1271; A61P 31/04; A61K 2039/505; C12N 9/22; C12N 15/74; C12N 15/111

(57) ABSTRACT

A pharmaceutical composition comprising an agent that increases the expression of a purR gene in a bacterium and a method of attenuating, preventing or treating a bacterial infection in a subject comprising administering to the subject an agent that increases the expression of a purR gene.

### FIELD OF THE INVENTION

The present invention relates in general to the attenuation, prevention and treatment of bacterial infection, more particularly to the prevention and treatment of bacterial infection involving overexpression of a purine biosynthesis repressor.

### BACKGROUND OF THE INVENTION

Antibiotic resistance in major human pathogens has reached a state of crisis, and the United Nations recently convened a historic summit to address global responses to this calamity. Renewed efforts to identify new drugs are urgently needed to address this increasingly serious public health threat. New classes of urgently-needed bacterial inhibitors will arise from innovative strategies that take into account the complexities of bacterial physiology during infection. This includes niche drugs that inhibit virulence factors in major human pathogens like S. aureus.

In humans, Staphylococcus aureus may exist as a commensal bacterium or as a pathogen. Data from the United States Centers for Disease Control and Prevention show that approximately one-third of the US population is colonized with S. aureus [1], and colonization with S. aureus is associated with increased risk of subsequent infection [2]. Infections caused by S. aureus range in severity from relatively minor skin and soft tissue infections through to invasive diseases such as pneumonia, infective endocarditis and osteomyelitis [3]. Strikingly, the magnitude of morbidity and mortality caused by S. aureus is highlighted by reports that, in the U.S., invasive infections by this bacterium cause more deaths than HIV [4].

That S. aureus can infect virtually any organ or tissue in the body is a reflection of its vast repertoire of virulence factors that contribute to bacterial pathogenesis through mechanisms involving tissue adherence [5,6], cellular intoxication [7-9], and immune modulation and deception [10,11]. Virulence factor expression in S. aureus is complex and coordinately regulated by multiple transcription factors, regulatory RNAs, two-component sensing systems and quorum-sensing [12-14]. Despite a wealth of knowledge on virulence regulation in S. aureus, there are still outstanding questions to be resolved, as novel mechanisms of virulence regulation are still being discovered, especially with regard to environmental or metabolic cues to which S. aureus responds [15].

Exposure to elevated temperatures, for example 42° C., a temperature frequently used to cure S. aureus of recombinant plasmids during mutagenesis procedures, can select for mutations in the S. aureus genome. Mutations in the global two-component regulator SaeRS have previously been isolated following mutagenesis [16], and mutations in the sae regulatory system show drastically reduced toxin production and have attenuated virulence [17-20]. Screening for unintended sae mutations is straight forward, as the mutants are easily identified as having reduced hemolytic activity on blood agar plates. Little is known, however, about other unintended secondary mutations that may be selected for in response to stress, especially those that may impact on the virulence potential of S. aureus.

### SUMMARY OF THE INVENTION

In one embodiment the present invention relates to a pharmaceutical composition comprising an agent that increases or upregulates the expression of a purine biosynthesis repressor (purR) gene in a bacterium.

In another embodiment, the present invention relates to a method of attenuating, preventing or treating an infection, disorder or lesion caused by bacteria in a subject. In one embodiment, the method comprising administering to the subject an agent that up-regulates or overexpresses a purR gene.

In one embodiment, the present invention is a method of attenuating, preventing or treating an infection or disorder in a subject caused by or associated with bacteria, comprising administering to the subject (a) an agent that increases the number of wild-type purine biosynthesis repressor (purR) protein in the bacteria, or (b) an interfering agent that that inhibits, competes, or titrates binding of a fibronectin binding protein in the bacteria to fibronectin.

In one embodiment of the method of attenuating, preventing or treating an infection or disorder in a subject caused by or associated with bacteria, the interfering agent that inhibits, competes, or titrates binding of the fibronectin binding protein in the bacteria to fibronectin comprises an antibody or antigen binding fragment that specifically recognizes or binds the fibronectin binding protein.

In another embodiment of the method of attenuating, preventing or treating an infection or disorder in a subject caused by or associated with bacteria, the agent that increases the number of wild-type PurR protein in the bacteria comprises one or more of: (a) a phage carrying copies of a wild-type purR gene; (b) a conjugative plasmid that can conjugate with the bacterium carrying copies of the wild-type purR gene; (c) a non-naturally occurring Clustered Regularly Interspaced Short Palindromic Repeats (CRISPR)-CRISPR associated (Cas) system comprising (i) a first regulatory element operable in the bacteria operably linked to at least one nucleotide sequence encoding a CRISPR-Cas system guide RNA that hybridizes with a target DNA sequence in a DNA molecule of the bacteria, and (ii) a second regulatory element operable in the bacteria operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the wild type purR gene in the bacteria; and, wherein the Cas9 protein and the guide RNA do not naturally occur together; or (d) wild-type purR protein or a fragment thereof conjugated to a carrier that transfers the wild-type conjugated purR protein or fragment thereof to the bacteria having the mutated purR gene.

In another embodiment of the method of attenuating, preventing or treating an infection or disorder in a subject caused by or associated with bacteria, the carrier is a liposome, a micelle, or a pharmaceutically acceptable polymer.

In another embodiment of the method of attenuating, preventing or treating an infection or disorder in a subject caused by or associated with bacteria, the bacteria includes a purR gene or a biological equivalent of the purR gene.

In another embodiment of the method of attenuating, preventing or treating an infection or disorder in a subject caused by or associated with bacteria according to any of the previous embodiments, the bacteria includes a mutant purR gene.

In another embodiment of the method of attenuating, preventing or treating an infection or disorder in a subject caused by or associated with bacteria according to any of the previous embodiments the bacteria is E. coli, S. aureus, or Bacillus subtilis.

In another embodiment of the method of attenuating, preventing or treating an infection or disorder in a subject caused by or associated with bacteria according to any of the previous embodiments the bacteria is S. aureus.

In another embodiment, the present invention is a method of inducing an immune response in or conferring passive immunity to bacteria in a subject in need thereof, the method comprising administering to the subject an effective amount of an agent that increases the number of wild type purine biosynthesis repressor (purR) protein or a functional fragment thereof in the bacteria, or an interfering agent that that inhibits, competes, or titrates binding of a fibronectin binding proteins in the bacteria to fibronectin.

In one embodiment of the method of inducing an immune response in or conferring passive immunity to bacteria in a subject in need thereof, the interfering agent that inhibits, competes, or titrates binding of the fibronectin binding protein in the bacteria to fibronectin comprises an antibody or antigen binding fragment that specifically recognizes or binds the fibronectin binding protein.

In another embodiment of the method of inducing an immune response in or conferring passive immunity to bacteria in a subject in need thereof, the agent that increases number of purR protein in the bacteria comprises one or more of: a phage carrying copies of the purR gene; a conjugative plasmid that can conjugate with the bacterium carrying copies of the purR gene; a non-naturally occurring Clustered Regularly Interspaced Short Palindromic Repeats (CRISPR)-CRISPR associated (Cas) system comprising (i) a first regulatory element operable in the bacterium operably linked to at least one nucleotide sequence encoding a CRISPR-Cas system guide RNA that hybridizes with a target DNA sequence in a DNA molecule of the bacterium, and (ii) a second regulatory element operable in the bacterium operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the wild type purR gene in the bacterium; and, wherein the Cas9 protein and the guide RNA do not naturally occur together; or wild-type purR protein or a fragment thereof conjugated to a carrier that transfers the wild-type conjugated purR protein or fragment thereof to the bacteria having the mutated purR gene.

In another embodiment of the method of inducing an immune response in or conferring passive immunity to bacteria in a subject in need thereof, the carrier is a liposome, a micelle, or a pharmaceutically acceptable polymer.

In another embodiment of the method of inducing an immune response in or conferring passive immunity to bacteria in a subject in need thereof according to any of the previous embodiments, the bacteria includes a purR gene or a biological equivalent of the purR gene.

In another embodiment of the method of inducing an immune response in or conferring passive immunity to bacteria in a subject in need thereof according to any of the previous embodiments, the bacteria includes a mutant purR gene.

In another embodiment of the method of inducing an immune response in or conferring passive immunity to bacteria in a subject in need thereof according to any of the previous embodiments, the bacteria is E. coli, S. aureus, or Bacillus subtilis.

In another embodiment of the method of inducing an immune response in or conferring passive immunity to bacteria in a subject in need thereof according to any of the previous embodiments, the bacteria is S. aureus.

In another embodiment, the present invention is a use of an agent an agent that increases the number of wild-type purine biosynthesis repressor (purR) protein or a functional fragment thereof in bacteria, or an interfering agent that that inhibits, competes, or titrates binding of a fibronectin binding proteins in the bacteria to fibronectin for attenuating, preventing or treating an infection or disorder in a subject caused by the bacteria having the mutant purR gene.

In another embodiment, the present invention is a use of an agent an agent that increases the number of wild-type purine biosynthesis repressor (purR) protein or a functional fragment thereof in bacteria, or an interfering agent that that inhibits, competes, or titrates binding of a fibronectin binding proteins in the bacteria to fibronectin for inducing an immune response in or conferring passive immunity to the bacteria having the mutant purR gene in a subject in need thereof.

In one embodiment of the use according to any of the previous embodiments, the interfering agent that inhibits, competes, or titrates binding of the fibronectin binding protein in the bacteria to fibronectin comprises an antibody or antigen binding fragment that specifically recognizes or binds the fibronectin binding protein.

In another embodiment of the use according to any of the previous embodiments, wherein the agent that increases the number of the wild-type purR protein or fragment thereof in the bacteria comprises one or more of: a phage carrying copies of the purR gene; a conjugative plasmid that can conjugate with the bacterium carrying copies of the purR gene; a non-naturally occurring Clustered Regularly Interspaced Short Palindromic Repeats (CRISPR)-CRISPR associated (Cas) system comprising (i) a first regulatory element operable in the bacterium operably linked to at least one nucleotide sequence encoding a CRISPR-Cas system guide RNA that hybridizes with a target DNA sequence in a DNA molecule of the bacterium, and (ii) a second regulatory element operable in the bacterium operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the wild type purR gene in the bacterium; and, wherein the Cas9 protein and the guide RNA do not naturally occur together; or a wild-type purR protein or a fragment thereof conjugated to a carrier that transfers the wild-type conjugated purR protein or fragment thereof to the bacteria having the mutated purR gene.

In another embodiment of the use according to any of the previous embodiments, the carrier is a liposome, a micelle, or a pharmaceutically acceptable polymer.

In one embodiment of the use according to any of the previous embodiments, the bacteria includes a purR gene or a biological equivalent of the purR gene.

In one embodiment of the use according to any of the previous embodiments, the bacteria includes a mutant purR gene.

In one embodiment of the use according to any of the previous embodiments, the bacteria is E. coli, S. aureus, or Bacillus subtilis.

In one embodiment of the use according to any of the previous embodiments, the bacteria is S. aureus.

In another embodiment, the present invention provides for an agent that increases the number of a wild-type purine biosynthesis repressor (purR) protein or a functional fragment thereof in bacteria.

In one embodiment of the present invention, the agent comprises one or more of: a phage carrying copies of the purR gene; a conjugative plasmid that can conjugate with the bacterium carrying copies of the purR gene; a non-naturally occurring Clustered Regularly Interspaced Short Palindromic Repeats (CRISPR)-CRISPR associated (Cas) system comprising (i) a first regulatory element operable in the bacterium operably linked to at least one nucleotide sequence encoding a CRISPR-Cas system guide RNA that hybridizes with a target DNA sequence in a DNA molecule of the bacterium, and (ii) a second regulatory element operable in the bacterium operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the wild type purR gene in the bacterium; and, wherein the Cas9 protein and the guide RNA do not naturally occur together; or a wild-type purR protein or a fragment thereof conjugated to a carrier that transfers the wild-type conjugated purR protein or fragment thereof to the bacteria having the mutated purR gene.

In another embodiment, the present invention provides for a hypervirulent bacterium that expresses a polypeptide encoded by a mutant purR gene. In one aspect, the polypeptide is any of SEQ ID NO.: 2 to SEQ ID NO.:15.

In another embodiment, the present invention provides for an isolated bacterium that overexpress a purR gene.

In another embodiment, the present invention provides for an isolated or recombinant protein comprising amino acid sequence of SEQ ID NO:2 to SEQ ID NO:15. In one aspect, the present invention provides for an isolated or recombinant nucleic acid that encodes the isolated or recombinant protein of EQ ID NO:2 to SEQ ID NO:15.

In another embodiment, the present invention provides for purR mutant polypeptide that confers hypervirulent phenotype in a bacterium. In one aspect of this embodiment, the purR mutant polypeptide comprises an amino acid sequence according to any one of SEQ ID Nos. 2 to 15.

In another embodiment, the present invention provides for a polypeptide that causes bacteria to aggregate (“clump”) in serum having fibronectin, wherein the polypeptide comprises an amino acid sequence according to any one of SEQ ID NO 2 to SEQ ID NO:15.

In another embodiment, the present invention provides for nucleic acid that encodes any of the polypeptides of claims 22 and 23.

In another embodiment, the present invention provides for a polypeptide that is at least 70% identical to the isolated or recombinant polypeptide of SEQ ID NO 2 to SEQ ID NO:15, and exhibits substantially equivalent biological activity to the polypeptide of SEQ ID NO 2 to SEQ ID NO:15.

In another embodiment, the present invention provides for a polypeptide that is encoded by a polynucleotide that hybridizes under stringent conditions to a complement of the nucleic acid that encodes the polypeptides of SEQ ID NO 2 to SEQ ID NO:15, and exhibits substantially equivalent biological activity to the polypeptide encoded by said nucleic acid.

### BRIEF DESCRIPTION OF THE DRAWINGS

The following figures illustrate various aspects and preferred and alternative embodiments of the invention.

FIG. 1. Disruption of purR causes cell clumping of S. aureus USA300. In (A), representative images of USA300, USA300 purR::ΦNΣ or the complemented purR::ΦNΣ mutant in culture tubes following growth in TSB with 10% (v/v) horse serum (TSB-S) for 3.5 h from a starting OD₆₀₀ equivalent of 0.03. In (B), graphical representation of the relative sedimentation of bacterial aggregates in cultures as grown in (A), reflected by the OD₆₀₀ values of the center of liquid cultures after sitting without shaking for 5 min following shaking at 37° C. for 3.5 hr. Data are mean±SEM of 4 independent experiments. *** indicate a p value <0.001, based on a one-way analysis of variance (ANOVA) with a Bonferroni post-test. In (C), the representative micrographs show bacterial cell clusters that arise during growth in TSB or TSB-S. White boxes define the region of interest that is depicted in the insets. Bars equal 40 μm. In (D), transmission electron micrographs are shown for S. aureus USA300 and the USA300 purR::ΦNΣ strain grown in the presence (TSB-S) or absence (TSB) of horse serum. The representative images depict cells at 11000× magnification and the bars equal 1 μm. Source data are provided as a Source Data file.

FIG. 2. The purR-dependent clumping phenotype requires fibronectin binding proteins and host fibronectin. In (A), cultures were grown in TSB or TSB-S for 3.5 h and then imaged on a wide field microscope at 40× magnification. White boxes define the region of interest that is depicted in the insets. Bars equal 40 μm. Representative images are shown. In (B), cultures were grown as in (A) and OD₆₀₀ was measured as described in the legend to FIG. 1 and in the Methods. Data shown are mean±SEM of 4 independent experiments. *** indicate a p value <0.001, based on a one-way ANOVA with a Bonferroni post-test. In (C), WT and the purR::ΦNΣ mutant were grown in TSB, TSB-S, TSB containing 10% v/v of various levels of Fn-depleted horse serum or Fn-depleted horse serum with the addition of eluted fibronectin (Fn depletion 3+Fn). Measurement of OD₆₀₀ of cultures to evaluate clumping was performed as described above. Data shown are mean±SEM of 5 independent experiments and 2 different Fn purifications. *** indicate a p value <0.001, based on a one-way ANOVA with a Bonferroni post-test. In (D), biofilm forming ability of indicated strains was measured after growth in TSB in a standard 96-well plate biofilm assay (see Methods). Data shown are mean±SEM of 4 experiments ** indicates a p value <0.01, *** indicate a p value <0.001, based on a one-way ANOVA with a Bonferroni post-test. Source data are provided as a Source Data file.

FIG. 3. purR mutations lead to transcriptional upregulation of the purine biosynthesis operon and fnbAB. (A) or purR::ΦNΣ mutant (B) containing a luciferase construct with the promoter sequence of fnbA or fnbB (see Methods) were grown in TSB and OD₆₀₀ and luminescence monitored. Data shown are mean±SEM of 3 experiments. In E, F and G, indicated strains were grown to OD₆₀₀ of 0.2, 0.6 or 1.0, total RNA was extracted and RT-PCR analysis performed for relative abundance of fnbA (C), fnbB (D) and purE (E) transcripts. All data were normalized to levels of rpoB and expressed as fold change using WT pALC (empty plasmid) as comparator at each OD₆₀₀ value. Data shown are mean±SEM of 4 independent experiments * indicates a p value <0.05, ** a p value <0.01 and *** indicate a p value <0.001, based on a one-way ANOVA with a Bonferroni post-test. Source data are provided as a Source Data file.

FIG. 4. A S. aureus purR mutant is hypervirulent via FnbAB. In (A), mice (9-12 per group) were infected with ˜1×10⁷ CFU of WT USA300, USA300 purR::ΦNΣ or complemented purR::ΦNΣ mutant and survival monitored over 72 h. *** indicates a p value <0.001, based on a Mantel-Cox test. In (B), animals were infected as in A, but with 2-2.5×10⁶ CFU, and (C) weight loss monitored daily for 48 h. ** p value <0.01, *** p value <0.001, based on a one-way ANOVA with a Bonferroni post-test. In (D), animals from B were sacrificed at 48 hours post infection (hpi), and heart, kidney and liver were harvested and bacterial burdens determined. Data shown are mean±SEM, * indicates a p value <0.05, ** p value <0.01, *** p value <0.001, based on a Student's unpaired t-test. In (E), 2 animals per bacterial strain were infected as in A, with approx. 1×10⁷ CFU, sacrificed at 24 hpi and organs harvested. Organs were paraffin embedded, sectioned and stained with H&E and a Gram stain. Representative images are shown. In (F), animals were infected as in A, with approx. 1×10⁷ CFU, with the inclusion of WTΔfnbAB and purR::ΦNΣΔfnbAB strains, and monitored for 72 h. *** indicates a p value <0.001, based on a Mantel-Cox test. In (G), the heart, kidney and liver from the animals infected in (E) were harvested at the point of sacrifice and bacterial burden determined. Data shown are mean±SEM, * indicates a p value <0.05, ** p value <0.01, *** p value <0.001, based on a Student's unpaired t-test. Source data are provided as a Source Data file.

FIG. 5. Anti-staphylococcal antibodies ameliorate purR hyper-clumping. A, WT or the purR::ΦNΣ mutant were grown in TSB, TSB-S or TSB with 10% v/v fresh human serum (TSB-HuS) for 3 h and relative clumping ability was measured using OD₆₀₀ as described above. Data shown are mean±SEM of 4 independent experiments. * indicates a p value <0.05, ** a p value <0.01 and *** indicate a p value <0.001, based on a one-way ANOVA with a Bonferroni post-test. WT (B) or the purR::ΦNΣ mutant (C) were grown in TSB-HuS (grey bars) or TSB with IgG-depleted human serum (HuS) (black bars) for 3 h and relative clumping ability measured as above. Data shown are mean±SEM of 4 experiments, with 4 donors. ** indicates a p value <0.01, *** indicates a p value <0.001, based on a one-way ANOVA with a Bonferroni post-test. In (D), whole cell lysates of WT, WT pfnbA or WTΔfnbAB were used for Western blots, with human serum (from donors in panels B and C) or a rabbit anti-Fnb serum (far right blot) used as a source of primary antibody. Source data are provided as a Source Data file.

FIG. 6. Vaccination with S. aureus expressing FnbAB is protective against a challenge with a purR mutant. A, vaccination scheme, with 6 animals per group. B, survival of animals challenged with 1×10⁷ CFU of WT or purR::ΦNΣ S. aureus following vaccination, as outlined in A. * indicates a p value <0.05, ** indicates a p value <0.01, based on a Mantel-Cox test, as compared to WT vaccinated, purR::ΦNΣ challenged animals. C, whole cell lysate of WT, WT pfnbA or WTΔfnbAB were used for a Western blot, with serum from vaccinated animals or a rabbit anti-Fnb serum (far right) used as a primary antibody.

FIG. 7. Disruption of purR has minimal effect on the S. aureus proteome or growth. a, total protein of USA300 and USA300 purR::ΦNΣ grown to exponential (OD₆₀₀ 0.6) or stationary phase (OD₆₀₀ 6.0) and separated on a 12% SDS polyacrylamide gel. b, growth curves of USA300, USA300 purR::ΦNΣ or complemented purR::ΦNΣ mutant in TSB. c, growth curves of USA300, USA300 purR::ΦNΣ and complemented purR::ΦNΣ mutant in TSB-S. d, relative expression of a selection of genes following growth in TSB to OD₆₀₀ of 1.0, measured by RT-PCR. All data were normalised to the levels of rpoB and the expression in the WT was set to 1.0. Data shown are mean±SEM of 4 samples. *** indicates a p value <0.001 based on a one-way ANOVA with a Bonferroni post-test. Source data are provided as a Source Data file.

FIG. 8. Disruption of purR results in a clumping phenotype in a variety of strains, but not in strain Newman. WT, purR::ΦNΣ or purR::ΦNΣ complemented constructs in strains RN6390 (a), MN8 (b), SH1000 (c) or Newman (d) were grown in TSB or TSB-S for 3.5 h. Cultures were imaged on a wide field microscope at 40× magnification (left panel) or absorbance measured (right panel). Bars equal 40 μm. Data shown are mean±SEM of 4 experiments. * indicates a p value <0.05, *** indicates a p value <0.001 based on a one-way ANOVA with a Bonferroni post-test. Source data are provided as a Source Data file.

FIG. 9. Passage of horse serum over a gelatin column removes soluble fibronectin. Horse serum was passaged over a gelatin sepharose column 3 times. Column flow through and elutions were separated on a 7% SDS polyacrylamide gel.

FIG. 10. S. aureus purR SNP mutant is hypervirulent. a, animals were infected IV with 1×10⁷ CFU of USA300 WT, purR::ΦNΣ or purR^{Q62P} mutant and monitored over 48 h. b, animals were infected IV with 1×10⁷ CFU of Newman WT or purR::ΦNΣ and monitored over 48 h.

FIG. 11. Mutations in purR are selected for during growth at elevated temperatures and in vivo during infection of mice. a, schematic of the P_{purE:gusA} construct that is integrated into the S. aureus genome. b, WT P_{purE::gusA} after 5 passages at 37° C. (left) and 42° C. (right), grown on TSA with tetracycline and X-gluc. In c-e, characterization of a clone of S. aureus USA300 containing a purRR^{96A} SNP isolated from the kidney of a mouse infected for 4 days with WT USA300. Strains were grown in TSB or TSB-S for 3.5 h, and cultures were imaged on a wide field microscope at 40× magnification (c) or relative clumping was measured using the OD₆₀₀ assay described above (d). Data shown are mean±SEM of 3 experiments. *** indicates a p value <0.001 based on a one way ANOVA with a Bonferroni post test. In (e), animals were infected IV with 1×107 CFU of WT, purR::ΦNΣ or purRR96A mutant and monitored over 96 h. *** indicates a p value <0.001, based on a Mantel-Cox test. Source data are provided as a Source Data file.

FIG. 12. Human IgG can alleviate purR dependent clumping in horse serum. Cultures of WT USA 300 or USA300 purR::ΦNΣ were grown in TSB, TSB-S or TSB-S with the addition of purified and concentrated human IgG (from serum IgG depletions shown in FIG. 5. Cultures were allowed to grow for 3 h from a starting OD₆₀₀ of 0.03 and the OD₆₀₀ values of the center of liquid cultures after sitting for 5 min were determined. Data shown are mean±SEM of 3-4 independent experiments with IgG from 3 different donors. * indicates a p value <0.05, ** a p value <0.01 and *** indicate a p value <0.001, based on paired student t test. Source data are provided as a Source Data file.

### DESCRIPTION OF THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +1-15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention. Embodiments defined by each of these transition terms are within the scope of this invention.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

“Hypervirulent bacteria” or “purR mutants” are used interchangeably to refer to bacteria, such as S. aureus, with mutations in the transcriptional repressor of purine biosynthesis, purR, which enhance the pathogenic potential of the bacterium due to aberrant up-regulation of fibronectin binding proteins (FnBPs).

As used herein, the terms “treating,” “treatment” and the like are used herein to mean obtaining a desired pharmacologic and/or physiologic effect. The effect may be prophylactic in terms of completely or partially preventing an infection, disorder or sign or symptom thereof and/or may be therapeutic in terms of a partial or complete cure for an infection, a disorder and/or adverse effect attributable to the infection or disorder.

To “prevent” intends to prevent an infection, lesion or disorder or effect in vitro or in vivo in a system or subject that is predisposed to the disorder or effect. An example of such is preventing an infection, lesion or disorder caused by of bacteria such as E. coli, B. subtilis, S. aureus among others, in a subject or system including infected with purR mutants of said bacteria.

The term “inhibiting, competing or titrating” intends a reduction in the formation of a protein/protein interaction (such as the interaction formed between FnBP and fibronectin) or a DNA/protein matrix.

An “interfering agent” intends an agent that any one or more of competes, inhibits, prevents, titrates a FnBP binding to fibronectin, or to any other protein that binds to FnBP. It can be any one or more of a chemical or biological molecule.

Examples of such molecules include: (1) small molecules that inhibit the binding activity of FnBP, (2) small molecules that compete with FnBP in fibronectin binding, (3) polypeptides such as peptide fragments of FnBP that compete with FnBP in binding fibronectin, or (4) antibodies or fragments thereof directed to FnBP.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, nasal administration, injection and topical application.

A “subject” refers to a member of the animal kingdom such as a mammal or a human. Non-human animals subject to the present invention are those subject to bacterial infections or animal models, for example, simians, murines, such as, rats, mice, chinchilla, canine, such as dogs, leporids, such as rabbits, livestock, sport animals and pets.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular/bacterial proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell/bacterium is a cell/bacterium that is separated from tissue or cells/bacteria of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

“Pharmaceutically acceptable carriers” refers to any diluents, excipients or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

“Plasmid” refers to an extra-chromosomal DNA molecule separate from the chromosomal DNA. Plasmids replicate extra-chromosomally inside a cell/bacterium and can transfer their DNA from one cell/bacterium to another by a variety of mechanisms. DNA sequences controlling extra chromosomal replication (ori) and transfer (tra) are distinct from one another; i.e., a replication sequence generally does not control plasmid transfer, or vice-versa.

A “conjugative plasmid” is a plasmid that is transferred from one organism, such as a bacterial cell, to another organism during a process termed conjugation. The term refers to a self-transmissible plasmid that carries genes promoting the plasmid's own transfer by conjugation. Cis-conjugative plasmids carry their own origin of replication, oriV, and an origin of transfer, oriT, and genes promoting the plasm id's own transfer by the conjugation process. Conjugation functions can be plasmid encoded, but some conjugation genes can be found in the bacterial chromosome or another plasmid and can exhibit their activity in trans to a separate plasmid that encodes, for example, the oriT sequence. Numerous conjugative plasmids are known, which can transfer associated genes within one species (narrow host range) or between many species (broad host range). Conjugation can occur between species classified as different at any taxonomic level—including in the extreme between domains, e.g. bacteria to eukaryotes.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

The term “protein”, “peptide” and “polypeptide” are used interchangeably and in their broadest sense refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

It is to be inferred without explicit recitation and unless otherwise intended, that when the present invention relates to a polypeptide, protein, polynucleotide or antibody, an equivalent or a biologically equivalent of such is intended within the scope of this invention. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or alternatively about 80% homology or identity and alternatively, at least about 85%, or alternatively at least about 90%, or alternatively at least about 95% or alternatively 98% percent homology or identity and exhibits substantially equivalent biological activity to the reference protein, polypeptide or nucleic acid. In another aspect, the term intends a polynucleotide that hybridizes under conditions of high stringency to the reference polynucleotide or its complement.

A polynucleotide or polynucleotide region (or a polypeptide or polypeptide region) having a certain percentage (for example, 70%, 80%, 85%, 90% or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 30% identity or alternatively less than 25% identity, less than 20% identity, or alternatively less than 10% identity with one of the sequences of the present invention. “Homology” or “identity” or “similarity” can also refer to two nucleic acid molecules that hybridize under stringent conditions to the reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“purR gene” is a gene encoding a repressor protein for purine nucleotide synthesis. The purR gene may be found in various bacteria, including for example E. coli, S. aureus, Bacillus subtilis among others. purR gene includes also biological equivalents of the purR gene found in E. coli, S. aureus, Bacillus subtilis.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)2, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-FnBP, for example, refers to a monoclonal or polyclonal antibody that binds and/or has an affinity to a particular protein. For example, “anti-FnBP” refers to an antibody that binds to the fibronectin binding protein. The specific antibody may have affinity or bind to proteins other than the protein it was raised against. For example, anti-FnBP, while specifically raised against the fibronectin binding protein, may also bind other proteins that are related either through sequence homology or through structure homology.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hyridoma proliferates and produces large amounts of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CD sequences derived from the germline of another mammalian species, such as a mouse, have been grafted onto human framework sequences. Thus, as used herein, the term “human antibody” refers to an antibody in which substantially every part of the protein (e.g., CDR, framework, CL, CH domains (e.g., CHI, Cm, CH3), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain human antibody may be at least 95%, or even at least 96%>, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germ line immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CD, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope. As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

Overview

Provided herein, are new agents and methods of preventing, attenuating or treating a bacterial infection by upregulating or over expressing, or by increasing the number of genes associated with purine synthesis repressor. The applicants have identified mutations that occur in the S. aureus purR gene in response to stress, including growth at elevated temperatures (i.e. 42° C.) and during infection of an immune competent subject. The function of purR in S. aureus has not been characterized, but the gene is homologous to those that encode the purine biosynthesis repressors in Bacillus subtilis and Escherichia coli; the applicants show here that mutations in purR result in upregulation of purine biosynthetic genes in S. aureus. The applicant has unexpectedly discovered that by upregulating or overexpressing the purR gene significantly decreases, or even eliminates, the formation of lesions due to bacterial infection.

Microbial infections, lesions and disease that can be treated by the compositions and/or methods of this invention include infection, lesions and diseases or disorders by bacteria carrying a purR gene, such as E. coli, B. subtilis, and S. aureus, among others.

In one embodiment the present invention relates to a pharmaceutical composition comprising an agent that increases or upregulates the expression of a purine biosynthesis repressor (purR) gene or the overexpression of the purR protein in a bacterium. The pharmaceutical compositions of the present invention may be used to prevent, attenuate or treat infections, disorders and/or lesions caused by bacteria that include a purR gene, or a gene equivalent to purR. The pharmaceutical composition may include one or more pharmaceutically acceptable carriers.

In another embodiment, the present invention relates to a method of attenuating, preventing or treating bacterial infection, disorder or and/or lesions in a subject. In one embodiment, the method includes administering to the subject an agent that upregulates or overexpresses a purR gene.

Agents that can be used in the pharmaceutical compositions and methods of the present invention include, for example: (a) a phage carrying copies of the purR gene, or carrying a regulatory element operable in a bacterium that increases the expression of the purR gene in the bacterium; (b) a conjugative plasmid that can conjugate with a bacterium carrying copies of the purR gene, or carrying a regulatory element operable in the bacterium that increases the expression of the purR gene; (c) a non-naturally occurring Clustered Regularly Interspaced Short Palindromic Repeats (CRISPR)-CRISPR associated (Cas) system comprising (i) a first regulatory element operable in the bacterium operably linked to at least one nucleotide sequence encoding a CRISPR-Cas system guide RNA that hybridizes with a target DNA sequence in a DNA molecule of the bacterium, and (ii) a second regulatory element operable in the bacterium operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the purR gene in the bacterium; and, wherein the Cas9 protein and the guide RNA do not naturally occur together; (d) a small molecule, such as a low molecular weight agent that, when introduced into a bacterium, results in upregulation in expression of purR polypeptide in the bacterium; or an agent that when introduced into a bacterium that expresses purR, inhibits or interferes with the expression of fibronectin binding proteins in the bacterium, such as an anti-fnbAB antibodies.

Routes of administration applicable to the compositions and methods of the invention include intranasal, intramuscular, intratracheal, subcutaneous, intradermal, topical application, intravenous, rectal, nasal, oral and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes. In general, routes of administration suitable for the methods of the invention include, but are not limited to, enteral, parenteral or inhalational routes.

Parenteral routes of administration other than inhalation administration include, but are not limited to, topical, transdermal, subcutaneous, intramuscular, intraorbital, intracapsular, intraspinal, intrasternal and intravenous routes, i.e., any route of administration other than through the alimentary canal. Parenteral administration can be conducted to effect systemic or local delivery of the agent. Where systemic delivery is desired, administration typically involves invasive or systemically absorbed topical or mucosal administration of pharmaceutical preparations.

The compounds of the invention can also be delivered to the subject by enteral administration. Enteral routes of administration include, but are not limited to, oral and rectal (e.g., using a suppository) delivery.

Methods of administration of the agent of the composition of the present invention through the skin or mucosa include, but are not limited to, topical application of a suitable pharmaceutical preparation, transcutaneous transmission, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In various embodiments of the methods of the invention, the agent will be administered orally on a continuous, daily basis, at least once per day (QD) and in various embodiments two (BID), three (TID) or even four times a day. Typically, the therapeutically effective daily dose will be at least about 1 mg, or at least about 10 mg, or at least about 100 mg or about 200-about 500 mg and sometimes, depending on the compound, up to as much as about 1 g to about 2.5 g.

Dosing of can be accomplished in accordance with the methods of the invention using capsules, tablets, oral suspension, suspension for intra-muscular injection, suspension for intravenous infusion, gel or cream for topical application or suspension for intra-articular injection.

Dosage, toxicity and therapeutic efficacy of compositions described herein can be determined by standard pharmaceutical procedures in cell cultures or experimental animals, for example, to determine the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Kits containing the agents and instructions necessary to perform in vitro and in vivo methods as described herein also are claimed. Accordingly, the invention provides kits for performing these methods which may include an agent of this invention as well as instructions for carrying out the methods of this invention such as collecting tissue and/or performing the screen and/or analyzing the results and/or administration of an effective amount of the agent as defined herein. These can be used alone or in combination with other suitable antimicrobial agents.

In another embodiment the present invention provides for hypervirulent bacteria that express a purR mutant polypeptide. The hypervirulent bacteria may express any of SEQ ID NO:2 to SEQ ID NO:15.

In another embodiment the present invention provides for an isolated bacterium that overexpress a purR gene. The isolated bacterium that overexpresses a purR gene can be used in the pharmaceutical compositions and methods of the present invention.

In one embodiment, the present invention provides for a purR mutant polypeptide that confers hypervirulent phenotype in a bacterium. In one aspect of the present invention, the purR mutant polypeptide comprises an amino acid sequence selected from SEQ ID Nos. 2 to 15. In another aspect, the purR mutant polypeptide comprises an amino acid sequence of SEQ ID NO:2 or SEQ ID NO:10.

In one embodiment the present invention provides for an isolated or recombinant protein comprising an amino acid sequence selected from SEQ ID NO:2 to SEQ ID NO:15.

In another embodiment the present invention provides for an isolated or recombinant nucleic acid that encodes the isolated or recombinant protein of the previous embodiment.

In another embodiment the present invention provides for a polypeptide that causes bacteria to aggregate (or “clump”) in serum. The polypeptide, in one aspect, comprises an amino acid sequence selected from SEQ ID NO 2 to SEQ ID NO:15.

The present invention includes also a polypeptide, protein or nucleic acid molecule that is at least 70% identical to any one of the polypeptides, proteins or nucleic acid molecules of the present invention and exhibits substantially equivalent biological activity to the reference protein, polypeptide or nucleic acid. Included in the present invention is also a polypeptide encoded by a polynucleotide that hybridizes under conditions of high stringency to a complement of a polynucleotide that encodes for any of the polypeptides of the present invention and exhibits substantially equivalent biological activity to the reference polypeptide.

The following example is intended to illustrate, but not limit the invention.

### EXAMPLES

### Example 1

Materials and Methods

Bacterial Growth Conditions

Bacterial strains and plasmids used in this study are listed in Table 1 and primers are listed in Table 2. E. coli was grown in Luria-Bertani (LB) broth and S. aureus was grown in tryptic soy broth (TSB) at 37° C., shaken at 200 rpm, unless otherwise stated. Where appropriate, media were supplemented with erythromycin (3 μg/mL), chloramphenicol (12 μg/mL), lincomycin (10 μg/mL), ampicillin (100 μg/mL) or tetracycline (3 μg/mL). Solid media were supplemented with 1.5% (w/v) Bacto agar.

PCR and Construct Generation

S. aureus strain USA300 LAC, cured of the 27-kb plasmid that confers antibiotic resistance, was used as the WT strain for mutant generation, unless otherwise stated. For mobilizing transposon insertion mutations into various genetic backgrounds, phage transduction was performed according to standard techniques. Phage lysate was prepared from the donor strain using phage 80a, recipient strains were infected and transductants selected using appropriate antibiotics. Insertions were confirmed by PCR. Markerless deletions were constructed using the pKOR1 system, as previously described⁵³. Briefly, upstream and downstream regions flanking the FnbAB genes were amplified with primers FnbAB Up F and Up R, and FnbAB Down F and Down R, respectively, using Phusion DNA polymerase and recombined into pKOR1. The resulting vector was passaged through RN4220 and subsequently introduced into strains of interest by electroporation. Genomic deletions were confirmed by PCR with primers hybridizing outside of the cloned area of interest. The purE promoter-glucuronidase fusion reporter was synthesised by Integrated DNA Technologies (IDT, Canada), and ligated into pLL29. pLL29 was transformed into RN4220 containing a plasmid encoding an integrase and later transduced into USA300 and derivatives⁵⁴. For complementation with WT purR or fnbA, the full-length genes were amplified using primers PurR F and PurR R and FnbA F and FnbA R, respectively, ligated into pALC2073 and recombinant plasmids transformed into E. coli. Plasmids were then passaged through RN4220, prior to transformation into the strain of interest. For insertion of fnb promoters into pGYlux, sequences were amplified from the USA300 genome with primer pairs pGYluxFnbA F and pGYluxFnbA R (for pGY:fnbA) and pGYluxFnbB F pGYluxFnbB R (for pGY::fnbB) respectively. Constructs were passaged through RN4220, prior to transformation into the strain of interest.

Clumping Assays

For measurement of clumping in serum (horse or human), overnight cultures in TSB were diluted to OD₆₀₀ 0.03 in 2 mL TSB or TSB with 10% (v/v) serum (TBS-S) in a 13 mL tube and grown at 37° C., with shaking at 200 rpm for 3.5 h. Cultures were allowed to sit without shaking for 5 min and the OD₆₀₀ of the middle of the culture was determined. The same cultures were imaged live on a brightfield Leica microscope at 40× magnification.

Fibronectin Removal

To remove fibronectin from horse serum, sterile, heat-inactivated horse serum was passaged over a column of gelatin sepharose (GE healthcare) (column bed volume of 5.5 mL) at approximately 1 mL/min and the flow through collected. The column was washed with approx. 20 mL of phosphate-buffered saline (PBS) and bound fibronectin was eluted with PBS+4 M urea. The column was re-generated as per manufacturer's instructions and the run-through from the first purification passaged again. A total of three passages over the column were performed and the fibronectin-free serum was sterilized by passage through a 0.22 μm filter. The different run troughs were used at 10% (v/v) in standard clumping assays, as described above.

Electron Microscopy

S. aureus strains were grown in TSB or TSB with 10% horse serum for 3.5 h, as previously described for clumping assays. The bacteria were then fixed overnight with a modified Karnovsky's fixative (2.5% glutaraldehyde+2% paraformaldehyde in 0.1M cacodylate buffer, pH 7.2). The fixed bacteria were embedded in a 1% agarose suspension and post-fixed with 1% (w/v) osmium tetroxide for 2 hours, followed by a 2-hour en bloc 0.5% uranyl acetate strain. Samples were then progressively dehydrated with 15-minute treatments of increasingly concentrated ethanol solutions (50, 70, 90, 95, 100%). After dehydration the samples were embedded in Epon-Araldite and ultrathin sections (70 nm) were cut and placed on nickel grids using an Ultracut microtome. The cut samples were surface stained for 15 minutes with 0.5% uranyl acetate and viewed with a Phillips 420 transmission electron microscope equipped with a Hamamatsu Orca 2 MPx HRL camera.

Preparation of Proteins

For examination of secreted protein profiles, strains were grown to the desired OD₆₀₀ in TSB and normalised to OD₆₀₀ of 6.0, pelleted by centrifugation, and supernatant mixed with 100% ethanol at a 1:3 ratio. Samples were incubated at −20° C. for 4-8 h and proteins pelleted at 5000×g for 30 min at 4° C. Pellets were re-suspended in 1:20^{th} of the original cultured volume in PBS and stored at −20° C. For whole cell lysate preparation, cells grown to the desired density were pelleted, washed once with PBS, re-suspended in 1:20th of the original volume in PBS with 400 μg lysostaphin and incubated at 37° C. for 1 h. Samples were passaged twice through a Cell-Disruptor (Constant Systems Ltd.) at 34 000 p.s.i., pelleted at 5000×g for 10 min and supernatant harvested. For mass spectrometry analysis of bacterial clumps, the purR::ΦNΣ mutant strain was grown in TSB with 10% (v/v) for 3 h at 37° C., and clumps allowed to settle at the bottom of the tube. Clumps were washed 3 times with PBS, dissolved in 1% SDS at 55° C. for 1 h and run on a 7% SDS polyacrylamide gel. Bands of interest were picked for LC-MS-MS analysis.

Western Blots

Strains used for Western blot analysis were the same as described above, with the additional deletion of protein A (spa) and sbi, to eliminate non-specific IgG interactions. Whole cell lysate was prepared as described above, mixed with 1× Laemmli buffer (60 mM Tris-HCl, pH 6.8, 2% SDS, 10% glycerol, 5% β-mercaptoethanol, 0.01% bromphenol blue), boiled for 10 min and separated on a 10% polyacrylamide gel. Following electrophoresis, proteins were transferred to a nitrocellulose membrane following standard protocols. Human, mouse or horse sera (1:500 dilution) or rabbit anti-FnbA antiserum⁵⁵ (1:500 dilution) were used as a primary antibody, and secondary antibody (conjugated to IRDye 800; Li-Cor Biosciences, Lincoln, Nebr.) was used at a 1:20,000 dilution. Membranes were scanned on a Li-Cor Odyssey Infrared Imager (Li-Cor Biosciences) and visualized using Odyssey Version 3.0 software.

Biofilm Assay

Biofilm assays were performed as described previously⁵⁶. Briefly, 200 μL of TSB supplemented with 0.4% w/v glucose was inoculated with a 1:100 dilution of an overnight culture. After static incubation at 37° C. for 16-22 h, cells were washed three times with PBS and fixed by drying at 42° C. Crystal violet (0.4%) was used to stain cells for 15 min, before being dissolved in glacial acetic acid (10%) and level of adhesion quantified by absorbance at 595 nm. Absorbance was normalized to the WT strain, which was set to 1.

Luciferase-Based Measurements of Fnb Promoter Activity

WT or purR::ΦNΣ strains carrying pGYlux constructs with the promoter of fnbA (pGY::fnbA) or fnbB (pGY::fnbB) were used. For luciferase measurements, overnight cultures grown in TSB with chloramphenicol were diluted to OD₆₀₀ of 0.01 in TSB with chloramphenicol and 200 μl added to a white optical 96 well plate (Thermo Fisher). Growth and luminescence were measured in a BioTek Synergy H4 plate reader at 37° C. with shaking. Data for both absorbance and luminescence was normalised to blank measurements for each time point.

RNA Extraction and RNAseq

S. aureus strains were grown overnight, subcultured to an OD₆₀₀ equivalent of 0.01 in TSB and grown to the desired growth phase. Cells equating to an OD₆₀₀ of 3.0 were harvested for each culture, and RNA extraction was performed by E.Z.N.A® total RNA kit (BioRad) according to the manufacturer's instructions with the addition of 0.25 μg/mL lysostaphin to the lysis solution. RNA purity was determined by visualisation on an agarose gel, and RNA concentration was determined by NanoDrop® ND-1000 UV-Vis spectrophotometer. cDNA preparation was performed using 500 ng of total cellular RNA reverse-transcribed using Superscript™ II reverse transcriptase (Invitrogen) according to the manufacturer's instructions. For each qPCR, 1 μg of cDNA was amplified in a Rotor-Gene 6000 (Corbett Life Science) using the iScript One-Step RT-PCR kit with SYBR Green (Bio-Rad). Gene expression for each sample was quantified in relation to rpoB expression. A standard curve was generated for each gene examined. For RNAseq, RNA was extracted as above, a library was constructed using an Illumina Script Seq RNA sequencing kit and sequenced on an Illumina MiSeq.

Genome Sequence Analysis

The nucleotide sequence of the purR gene was downloaded from 8207 S. aureus available genome sequences or assemblies from the NCBI database (1 Dec. 2017).

Sequences were translated and amino acids aligned to the USA300 LAC purR sequence using MEGA 7. For strains with sequence changes as compared to USA300 FPR3757, the information available was compiled into Table 5.

Ethics Statements

Human blood was obtained from healthy adult volunteers, with written permission and in compliance with protocol 109059 approved by the Office of Research Ethics at the University of Western Ontario. All animal experiments were performed in compliance with guidelines set out by the Canadian Council on Animal Care. All animal protocols (protocol 2017-028) were reviewed and approved by the University of Western Ontario Animal Use Subcommittee, a subcommittee of the University Council on Animal Care.

Human Serum Antibody Removal

Blood was allowed to clot for 30 min at RT, centrifuged at 400×g for 10 min (no brake) and serum harvested. Serum was filtered through a 0.22 μm filter and heat-inactivated for 1 h at 56° C. For removal of antibody, 4 mL serum was loaded on a HiTrap protein A column (GE healthcare) at 1 mL/min, followed by a 15 min wash (20 mM sodium phosphate, pH 7) at 1 mL/min. Antibody was eluted (0.1 M sodium citrate, pH 4) in 3 fractions (1.5 mL/each) at 1 mL/min. Serum used for clumping assays was passaged through the column twice. Eluted IgG filtered through a 0.22 μm filter, concentrated with an Amicon ultra-50 centrifugal filter and added to clumping assays containing 10% (v/v) horse serum.

Mouse Infections

6-8 week old female BALB/c mice (Charles River laboratories) were injected via tail vein with 100 μL of bacterial culture, containing 1×10⁶-1×10⁷ CFU of bacteria, as described in the text. To prepare the bacteria, strains were grown to OD₆₀₀ 2-2.5 in TSB, washed twice with PBS and re-suspended to the desired OD₆₀₀ in PBS. Infections were allowed to proceed for up to 96 h before animals were euthanized, or when they met guidelines for early euthanasia. Organs were harvested in PBS+0.1% Triton X-100 (Sigma), homogenised in a Bullet Blender Storm (Next Advance, Troy, N.Y.), using 2 runs of 5 min at setting 10, and metal beads. Dilutions of organ homogenates were plated on TSA for CFU enumeration. For vaccination studies, bacteria were grown to OD₆₀₀ of approx. 0.6, bacteria washed as above, heat killed at 85° C. for 15 min and 100 μL, equivalent to approx. 1×10⁸ CFU, were injected intraperitoneally (IP). For challenge post vaccination, infections were as outlined above.

Statistical Analysis

Statistical analyses were performed with GraphPad Prism software v5.0 or v 7.0.

Results

S. aureus purR Mutants Vigorously Clump During Growth in Serum

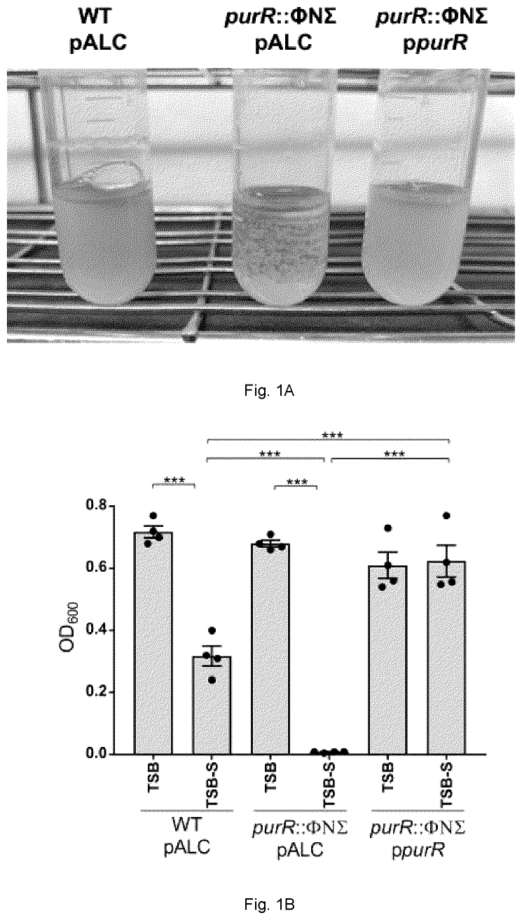

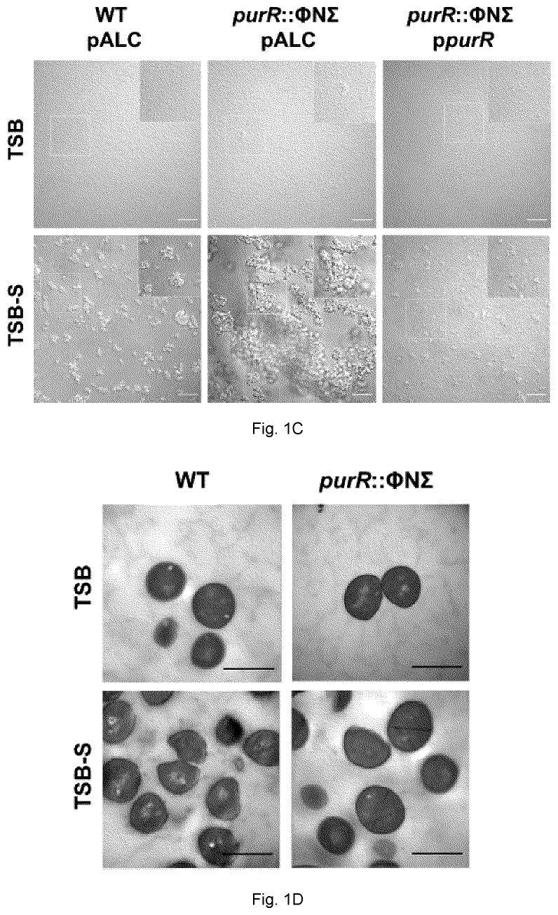

We generated deletion mutations in iron-regulated genes and test mutants for growth in chemically defined media (e.g. RPMI-1640) containing 10% v/v horse serum (HS) to induce iron starvation. Over time, we noted that a number of mutants, in the USA300 genetic background, would clump vigorously when grown the presence of HS, a trait not observed for WT USA300. The hallmark of this phenotype was that, during growth, visibly large clumps would appear in the culture tube and, when the culture tube was allowed to sit without shaking, the clumped material would settle to the bottom of the tube within minutes. This response was independent of iron starvation as robust clumping occurred when the bacteria were grown in tryptic soy broth, an iron replete medium, containing 10% v/v HS (TSB-S). To investigate this phenotype further, we performed whole genome sequencing on one of these clumping mutants and identified a non-synonymous single nucleotide polymorphism (SNP) in the purR gene [wild type PurR protein is SEQ ID NO:1, the nucleotide sequence of wild type PurR gene is SEQ ID NO: 16] causing a Q52P mutation (purR^{Q52P}) [SEQ ID NO:2]. The purR gene is homologous to those encoding the purine biosynthesis repressors in E. coli and B. subtilis but, to date, has not been studied in S. aureus. We independently discovered a second clumping mutant while generating a completely separate markerless deletion in the USA300 genome. We PCR-amplified the purR gene and discovered it carried a deletion of a guanine at position 682 of the gene, causing a frameshift in the protein after V229 [SEQ ID NO:3]. To confirm that loss of purR indeed correlated with the hyper-clumping phenotype, we mobilized the purR::ΦNΣ mutation from the Nebraska transposon mutant library²¹ into our laboratory USA300 strain (hereafter referred to as purR::ΦNΣ). The purR::ΦNΣ strain demonstrated similar clumping to the SNP-containing strain and the phenotype could be fully complemented by providing purR in trans on a multi-copy plasmid (referred to as ppurR) (FIG. 1A). Given that cultures containing clumped bacteria, when allowed to sit without shaking, rapidly clarify due to sedimentation of the cells in culture tubes, we developed an assay to quantitate relative clumping by measuring the culture optical density (see methods). This analysis detected a significant decrease in OD₆₀₀ values for both WT USA300 and purR::ΦNΣ in TSB-S, when compared to TSB alone. However, bacterial sedimentation (i.e. clumping) was greatly enhanced for purR::ΦNΣ in serum as compared to WT USA300 (FIG. 1B). Furthermore, these measurements confirmed that provision of purR in trans completely reversed the clumping phenotype (FIG. 1B).

To study the hyper-agglutination phenotype further, we used brightfield microscopy to examine the cells grown in TSB or TSB-S. WT USA300 and the complemented purR::ΦNΣ mutant in TSB formed only small ‘grape-like’ clusters of 2-4 cells, as expected for S. aureus. In contrast purR::ΦNΣ formed aggregates comprised of greater numbers of cells, including some noticeably larger clusters that were not observed for WT bacteria (FIG. 1C, top panels). Consistent with what is known of the interaction of S. aureus with serum proteins^{22,23}, USA300 and the purR::ΦNΣ complemented strains, grown in TSB-S as compared to TSB alone, formed larger cell clusters due to aggregation of the bacteria through binding of serum proteins (FIG. 1C, bottom panels). In contrast, bacterial aggregation was greatly exaggerated for purR::ΦNΣ, where aggregated masses of bacteria took up majority of the field of view (FIG. 1C, bottom panel) and undoubtedly related to the macroscopic sedimentation seen in liquid cultures.

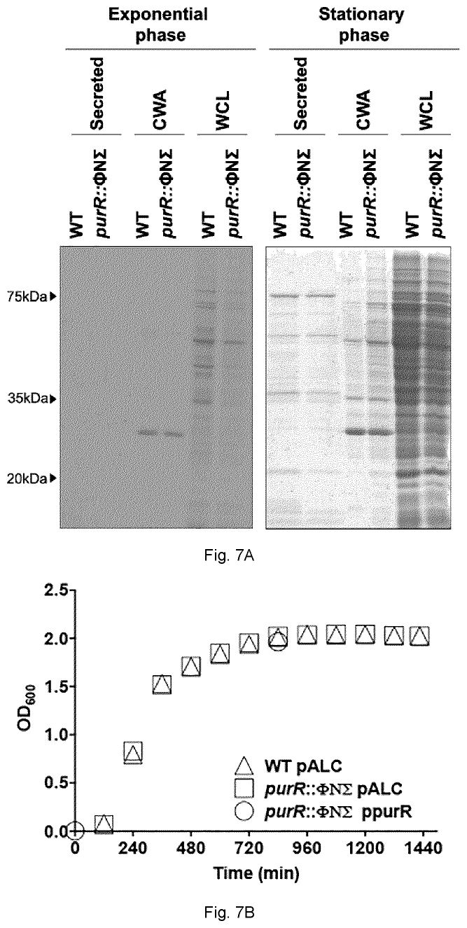

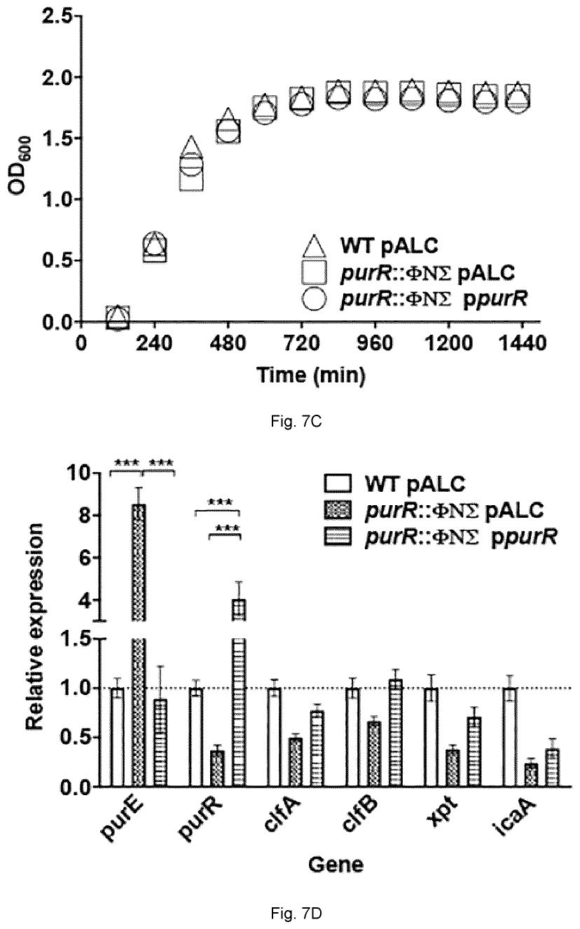

To assess whether cell clumping could be caused by cell division defects in the purR::ΦNΣ background, we performed transmission electron microscopy of WT or purR::ΦNΣ mutant cells, grown in TSB or TSB-S. For both strains, irrespective of culture conditions, division septa were visible and the apparent cell morphology did not differ, indicating cell division defects were not present in purR::ΦNΣ (FIG. 1D). We therefore next hypothesized that the robust aggregation of purR bacteria was mediated by specific bacterial factors. Interestingly, no discernible differences in protein profiles or growth were observed between WT and purR::ΦNΣ bacteria at various growth phases (FIG. 7A-7C). Transcriptional analysis was also performed by RNAseq on mid-exponential (OD₆₀₀=1.0) phase cultures grown in TSB. This analysis showed the genes of the purine biosynthesis pathway were elevated in the purR::ΦNΣ strain, as compared to the WT, however, few other differences could be detected between the two genotypes (Table 3). These findings were validated through RT-PCR for a number of genes that were differentially affected, and the results established that the relative expression patterns agreed with the RNAseq data, where purE, the first gene in the purEKCSQLFMNHD purine biosynthetic operon, demonstrated the greatest transcriptional increase (FIG. 7D). Altogether, these data demonstrate that purR regulates the purine biosynthesis pathway of S. aureus and inactivation of purR leads to exaggerated serum-dependent cell clustering. However, these analyses failed to identify an obvious effector responsible for the clumping phenotype.

Serum Clumping Requires Fibronectin Binding Proteins

S. aureus can produce two FnBPs, encoded by tandemly duplicated fnbA and fnbB genes. FnBPs, archetypal members of the microbial surface components recognizing adhesive matrix molecules (MSCRAMM) family, have a multi domain structure (5, 57).

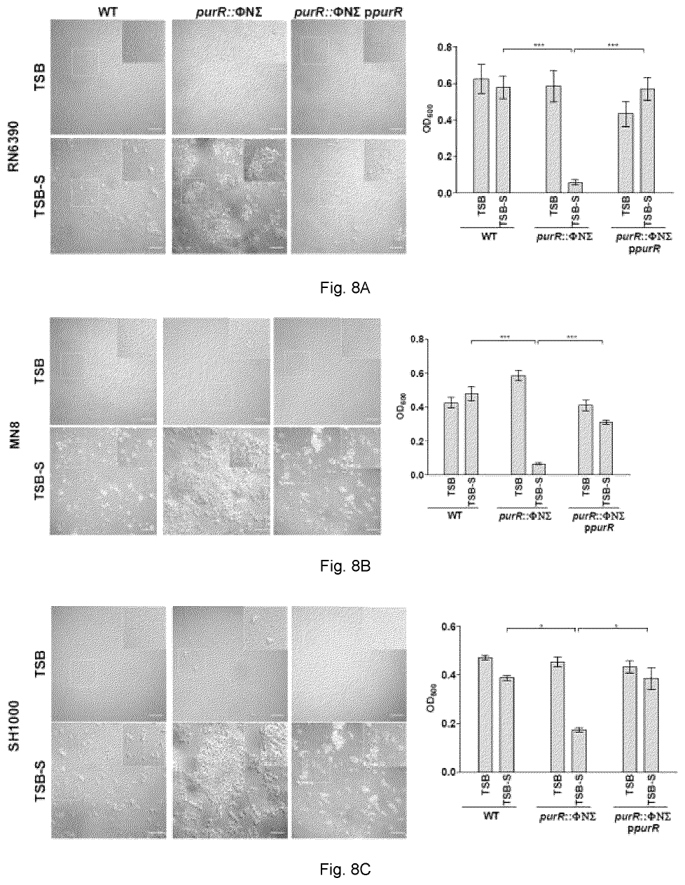

As an alternative approach to elucidate the mechanism by which purR bacteria hyper-aggregate we analyzed whether the purR phenotype was conserved across different S. aureus backgrounds. To this end, we transduced the purR::ΦNΣ mutation into S. aureus strains RN6390, SH1000, MN8 and Newman and complemented each mutant. Similar to USA300, growth of the RN6390, SH1000 and MN8 purR mutants in TSB-S demonstrated vigorous cell clumping and, for each strain, provision of purR in trans complemented the phenotype (FIGS. 8A-8C). In contrast, the Newman purR::ΦNΣ mutant failed to hyper-aggregate in the presence of HS and was indistinguishable from WT Newman when grown in either TSB or TSB-S (FIG. 8D). Of note, strain Newman expresses mutated fibronectin binding proteins (FnBPs; FnbA and FnbB) that, unlike in other S. aureus strains, are not cell wall anchored²⁴, suggesting that cell wall anchored FnbA/B may be required for hyper-clumping.

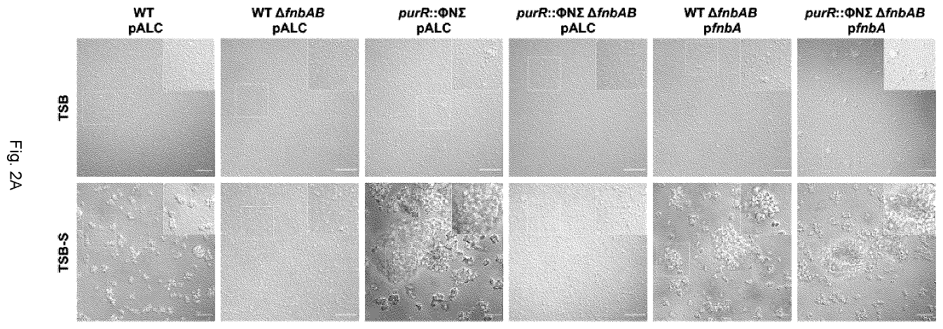

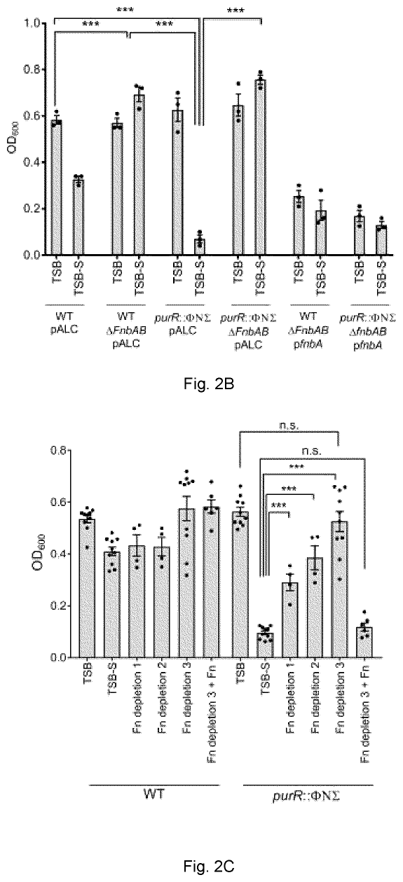

To directly test the involvement of the FnBPs in purR-dependent clumping, we engineered, in WT and purR::ΦNΣ USA300 bacteria, markerless deletions of the tandemly-duplicated fnbA and fnbB genes. Growth of the resulting purR::ΦNΣ fnbA/B mutants in TSB and TSB-S did not differ from that of WT USA300, and, notably, serum-dependent hyper-clumping did not occur (FIGS. 2A, 2B). Indeed, the USA300ΔfnbAB construct exhibited less serum-dependent clumping than WT, demonstrating the importance of these proteins in normal interactions of S. aureus with serum components (FIG. 2A). Of note, complementation of the ΔfnbAB mutants with fnbA on an overexpression plasmid resulted in exaggerated clumping during growth in TSB without serum (FIGS. 2A, 2B), likely due to the increased number of homophilic interactions between FnbA molecules, which have previously been reported to contribute to bacterial aggregation²⁵. Overall, these data indicate the hyper-clumping phenotype due to purR inactivation can be observed in a wide range of S. aureus strains and requires cell wall-anchoring of the FnBPs.

Serum Clumping by purR Mutants Requires Fibronectin

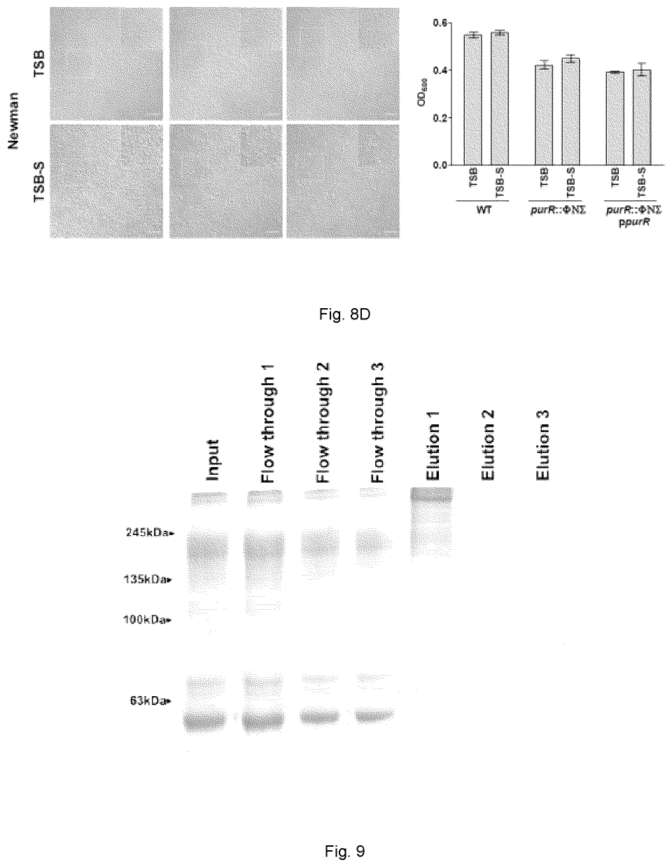

The multifunctional S. aureus FnBPs bind to fibrinogen, fibronectin and elastin⁵. To determine which serum component was involved in the clumping phenotype, we allowed purR::ΦNΣ bacteria to grow in TSB-S and form clumps. We isolated the clumped material and used mass spectrometry to identify enriched serum proteins that copurified with the bacteria (see methods). These analyses revealed only one protein, fibronectin (Fn) from Equus ferus przewalskii (Mongolian wild horse) was significantly enriched in purR::ΦNΣ derived samples. To confirm the involvement of Fn for purR-dependent hyper-clumping, soluble Fn was removed from horse serum by serial passage over a gelatin sepharose column (FIG. 9). When the Fn-depleted serum was used in clumping assays, we observed that Fn removal decreased the hyper-clumping phenotype of the purR::ΦNΣ mutant in a concentration dependent manner (FIG. 2C). Furthermore, reconstitution of the Fn-depleted serum with the purified horse Fn restored purR-dependent clumping to normal levels (FIG. 2C). Together these data demonstrate the purR-dependent clumping in serum requires S. aureus FnBPs and host Fn.

S. aureus purR Mutants Demonstrate Enhanced Biofilm Formation

The clustering of purR::ΦNΣ mutant cells in TSB, coupled with the dependency of the aggregation phenotype on FnBPs lead us to hypothesise that purR::ΦNΣ mutant bacteria were better able to initiate biofilm formation. To test this, we cultured the purR::ΦNΣ and fnbAB mutants in a standard 96-well plate biofilm assay. The purR::ΦNΣ mutant indeed formed increased biofilm as compared to WT USA300 (FIG. 2D), and this phenotype could be eliminated by the deletion of fnbAB in the purR::ΦNΣ background (FIG. 2D). Moreover, deletion of the fnbAB genes eliminated any differences between WT and purR::ΦNΣ cells and diminished biofilm formation altogether. Conversely, overexpression of fnbA from a plasmid enhanced biofilm formation irrespective of purR. These data indicate the clustering of the purR::ΦNΣ mutant cells augments biofilm formation and this requires FnBP expression.

PurR Represses Transcription of the purE Operon and fnbAB

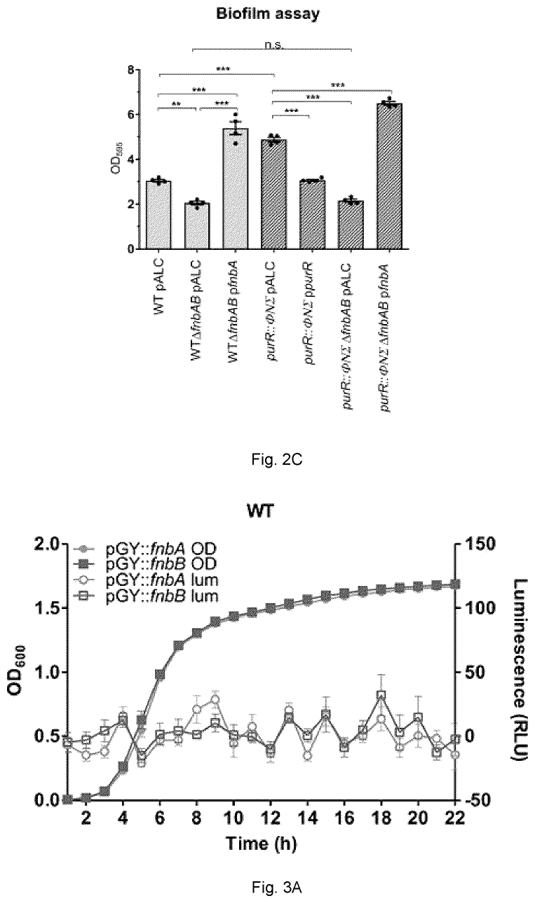

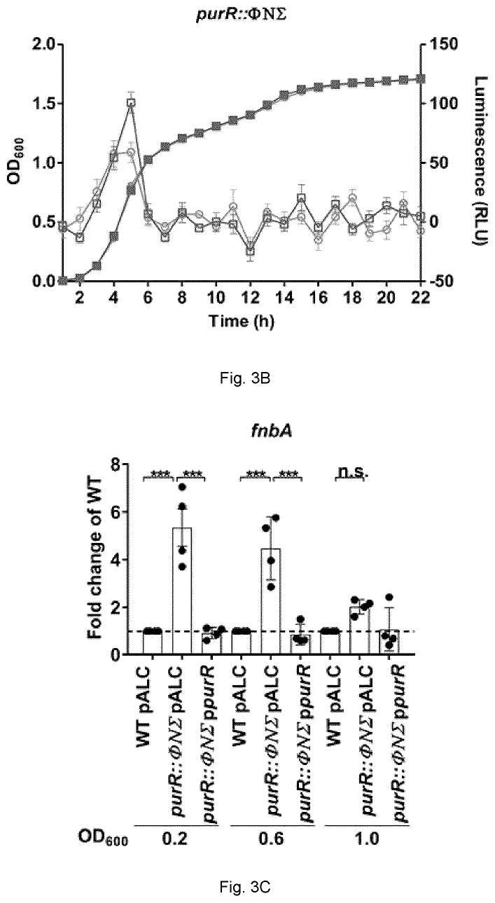

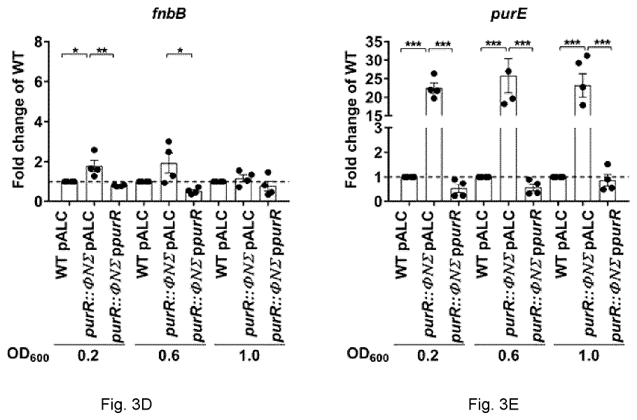

How inactivation of purR, a regulator of pur gene transcription, is connected to FnBP function and/or expression was not understood, as our RNAseq analysis failed to detect changes in either fnbA or fnbB transcript levels at culture densities of OD₆₀₀ of 1.0. However, looking into PurR gene regulation in Bacillus and Lactococcus gave us a clue that S. aureus PurR may regulate expression of fnbAB genes in S. aureus, but not during growth conditions we had thus far tested. Studies in B. subtilis and Lactococcus lactis have identified conserved sequence motifs in promoter regions, named PurBoxes, where PurR binds. Single or double PurBoxes can be present, and double PurBoxes are often palindromic, but all contain a central conserved CGAA motif^{26,27} (Table 6). Analysis of the USA300 genome identified a sequence similar to that of B. subtilis and L. lactis upstream of the purE and purA genes in S. aureus USA300 (Table 6) and, not surprisingly, these genes are upregulated in the purR::ΦNΣ strain (see Table 3). Remarkably, a similar putative PurR-binding sequence was also present upstream the fnbA and fnbB genes (Table 6). To determine whether transcription from the FnBP-encoding genes is influenced by PurR we generated plasmids carrying the fnbA and fnbB promoters fused to a promoterless lux-gene construct and monitored bioluminescence in WT or purR::ΦNΣ bacteria. Bioluminescence could not be detected above background levels in WT cells, presumably due to low levels of transcription from the fnbA/B promoters (FIG. 3A). In contrast, bioluminescence was detected for both the fnbA and fnbB promoter constructs in the purR::ΦNΣ mutant, where luminescence peaked at a culture density of OD₆₀₀ 0.5-0.6 (FIG. 3B). Based on these findings, we investigated transcript levels for fnbA and fnbB at early growth phases by RT-PCR. Relative to WT, fnbA transcripts were upregulated in the purR::ΦNΣ mutant, at culture densities as low as OD₆₀₀ of 0.2 (FIG. 3C), and steadily decreased as the culture density increased. Consistent with our RNAseq analysis, no significant differences in fnbA transcripts were present between the WT and the purR::ΦNΣ mutant at an OD₆₀₀ of 1.0. Of note, fnbB transcripts were only elevated in the purR::ΦNΣ mutant at OD₆₀₀ of 0.2 (FIG. 3D). Consistent with de-repression due to the absence of its regulator/repressor, and concordant with our previous data, purE transcripts were up regulated at all time points tested (FIG. 3D). Taken together, these data indicate transcriptional up-regulation of fnbA in the purR::ΦNΣ mutant at early growth phases, likely due to lack of binding of the PurR repressor to the upstream promoter-operator sequences.

S. aureus purR Mutants are Hypervirulent

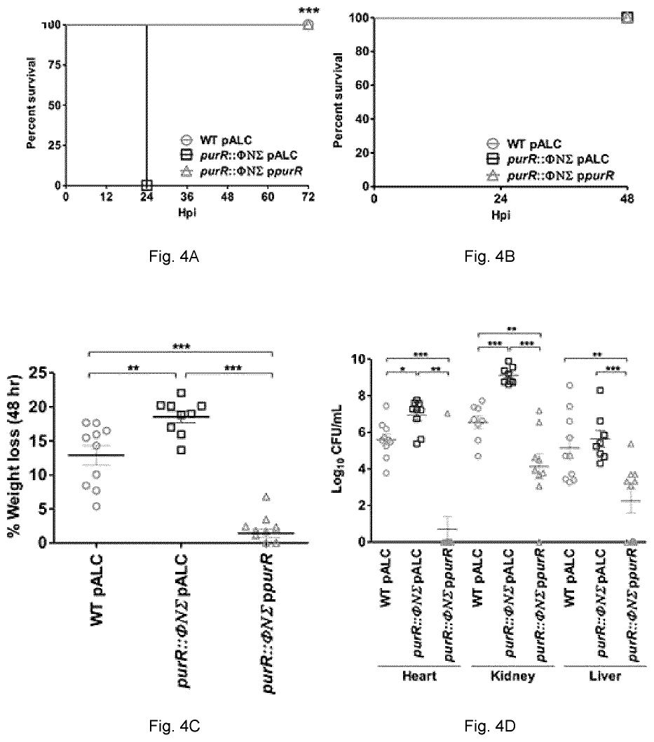

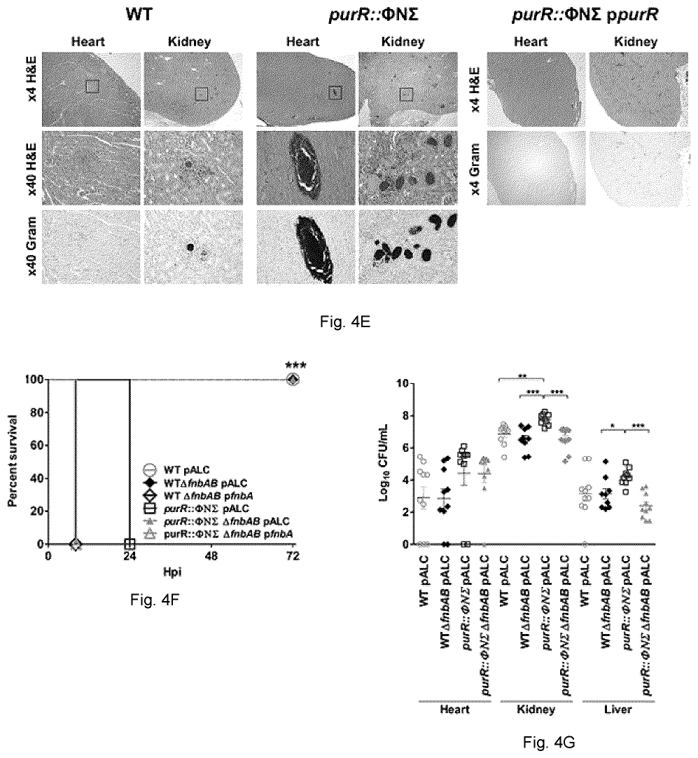

Given the strong Fn-binding phenotype associated with the S. aureus purR mutant, we next chose to evaluate the virulence potential of the mutant in a systemic mouse infection model. Mice were infected via the tail vein with WT USA300, the purR::ΦNΣ mutant, and the purR::ΦNΣ mutant complemented with ppurR at a dose of ˜1×10⁷ CFU. Remarkably, 100% of the mice infected with the purR::ΦNΣ mutant met humane endpoint criteria by 24 hpi, whereas 100% of the mice infected with either the WT or complemented mutant survived past 72 hpi (FIG. 4A). The purR^{Q52P} demonstrated the same hypervirulent phenotype as the purR::ΦNΣ mutant, as mice infected with this mutant required sacrifice at approximately 24 hpi (FIG. 10A).

In subsequent experiments, we tested the effect of a lower dose of the purR::ΦNΣ mutant and found that infection with ˜2×10⁶ CFU allowed murine survival up to 48 hpi (FIG. 4B). At 48 hpi, we observed significantly greater weight loss and increased bacterial burdens in mice infected with the purR::ΦNΣ mutant, when compared to those infected with the WT (FIGS. 4C and 4D). The mice infected with the complemented strain showed statistically significant decreases in weight loss and bacterial burden, even compared to mice infected with WT. In fact, near complete clearance of the complemented bacteria was observed in the heart (FIG. 4D).

Histopathological analysis of animals infected with high dose WT S. aureus (˜1×10⁷ CFU) for 24 h demonstrated lesions predominantly in the heart and the kidney (FIG. 4E). Animals infected with the purR::ΦNΣ mutant had larger and more frequent lesions in both the heart and kidneys (Table 3), with multifocal necrotic areas, often centered on discrete groups of Gram-positive bacteria (FIG. 4E). Complementation of the purR::ΦNΣ mutant almost completely eliminated the formation of lesions (FIG. 4E), concurring with the decreased bacterial burden previously observed and consistent with the use of an overexpression plasmid.

To confirm the role of fnbAB in the purR hypervirulence phenotype, we infected mice with the ΔfnbAB mutant, in either the WT or purR::ΦNΣ background. While purR::ΦNΣ infected animals required sacrifice by 24 hpi, as previously observed, the deletion of fnbAB in that background completely ablated the hypervirulent phenotype (FIG. 4F). Of note, infections with strains carrying an fnbA overexpression plasmid, indifferent of the purR background, resulted in very rapid effects on animal health, and animals required euthanasia by approx. 6-8 hpi (FIG. 4F). This demonstrates the profound effects of aberrant fnb expression, suggesting that even transient upregulation of FnBPs has a severe impact on disease severity in a systemic mouse model. Bacterial burden in the heart, kidneys and liver of the remaining groups was in agreement with the survival data, with increased numbers of bacteria for the purR::ΦNΣ strain, but not for the purR::ΦNΣ ΔfnbAB strain, compared to WT (FIG. 4G) (CFU for pfnbA carrying strains were not determined). Of note, no difference in survival or bacterial burden was seen between the WT and WT ΔfnbAB strains, indicating that while these proteins are not required for pathogenesis of WT USA300 in a systemic model of infection, they are indispensable for the hypervirulent phenotype of the purR::ΦNΣ mutant. In agreement with the requirement for FnbAB for hypervirulence, a purR::ΦNΣ mutant in strain Newman was not hypervirulent in this model (FIG. 10B). Taken together, these data demonstrate mutations in purR result in a hypervirulent phenotype in mice, in a FnBP-dependant manner.

Mutations in purR Occur at Elevated Temperatures and In Vivo

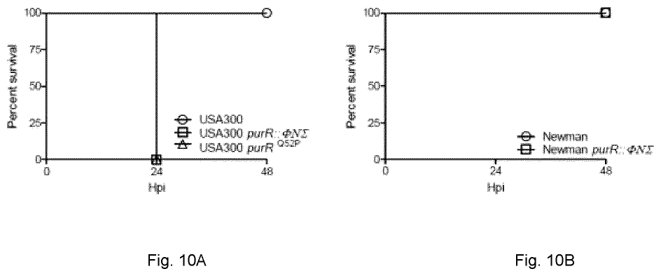

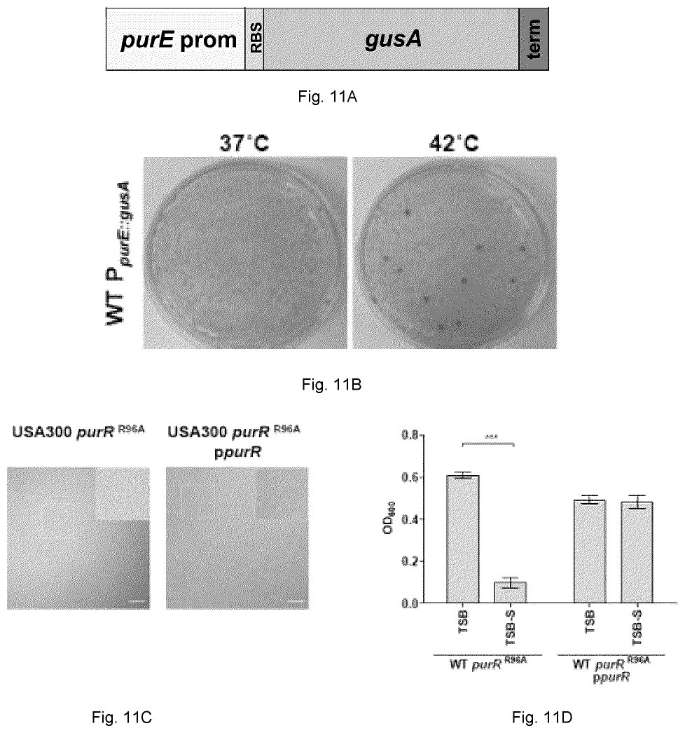

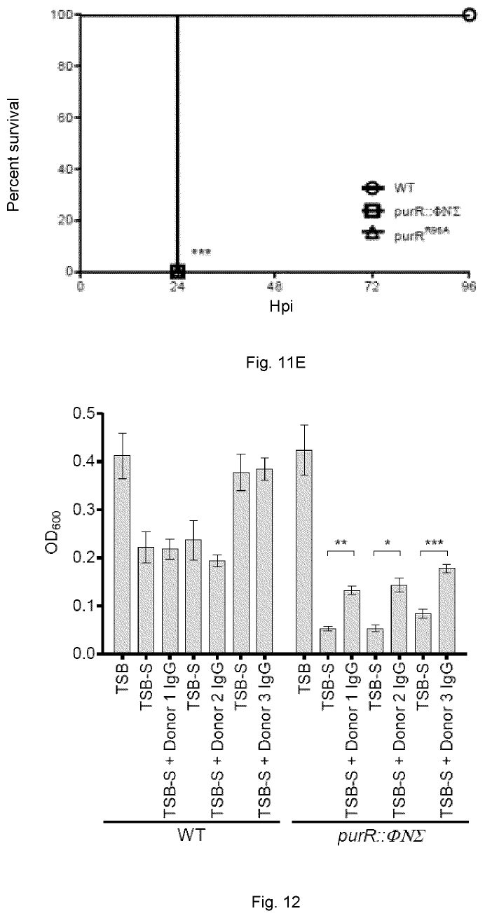

The purR^{Q52P} [SEQ ID NO.:2] SNP and the purR^{V229frameshift} [SEQ ID NO.:3] SNP were isolated following allelic replacement mutagenesis techniques, a process that has previously been reported to select for mutations in the virulence regulatory genes saeRS¹⁶. Plasmids for allelic replacement are often temperature sensitive and curing of plasmids following homologous recombination necessitates growth at elevated temperatures. To investigate if exposure to high temperatures also selects for purR mutations in S. aureus, we constructed a reporter strain that colorimetrically identify purR mutants. Given that the promoter of the purine biosynthetic operon (purEKCSQLFMNHD) was highly expressed in a purR mutant background, we fused the promoter of this operon to a promoterless gusA gene, encoding β-glucuronidase (referred to as P_{purE::gusA}) (FIG. 11A), and inserted this fusion into the S. aureus genome using a published procedure (see Methods). When cultured on X-Gluc-containing solid media, USA300 P_{purE::gusA} colonies were pale yellow while the USA300 purR::ΦNΣ strain carrying the genomic reporter were dark blue; this indicates that the reporter was capable of identifying purR mutants in culture. To define whether we could identify naturally-occurring purR mutants, we cultured USA300 P_{purE::gusA} at either 37° C. or 42° C., with daily passage for 5 days. Blue colonies were only detected from cultures grown at 42° C. (FIG. 11B), at a frequency of approximately 0.1-1.0% following 5 days of passaging (FIG. 11B). Sequencing of the purR gene from select blue colonies identified a variety of additional mutations in purR (Y71Stop [SEQ ID NO.:4], V156E [SEQ ID NO.:5], S172Stop [SEQ ID NO.:6], H225D [SEQ ID NO.:7], S230Stop [SEQ ID NO.:8], Q240Stop [SEQ ID NO.:9]).

The in vivo environment also presents a strong selection pressure on bacteria. Therefore, we were interested to determine if passage of WT S. aureus through mice would select for purR mutants. Unfortunately, for unknown reasons, the USA300 P_{purE::gusA} construct was lost from the genome without antibiotic selection in vivo. Therefore, we tested colonies recovered from the organs of mice infected with WT USA300 for 4 days for clumping in TSB-S (in a 96-well plate format). Potential mutants were phenotypically confirmed in a tube assay and the purR gene sequenced. A mutant with a R96A [SEQ ID NO.:10] SNP was identified from an infected kidney, and demonstrated cell clustering in TSB (FIG. 11C) and clumping in TSB-S (FIG. 11D), similar to what we previously observed with the purR::ΦNΣ mutant. The phenotype could be complemented by the introduction of ppurR, indicating the SNP was solely responsible for the observed phenotype (FIGS. 11C, 11D). It was of interest whether SNPs recovered from murine infections also displayed the characteristic hypervirulence we described here. Infection of mice with the R96A [SEQ ID NO.:10] SNP resulted in the same hypervirulent phenotype as the purR::ΦNΣ mutant (FIG. 11E), with animals requiring sacrifice within 24 hpi. Taken together, these data indicate purR mutations can be selected in response to stress, including due to elevated temperature and during murine infection.

Anti-FnBP Antibodies Ameliorate purR Mutant Clumping

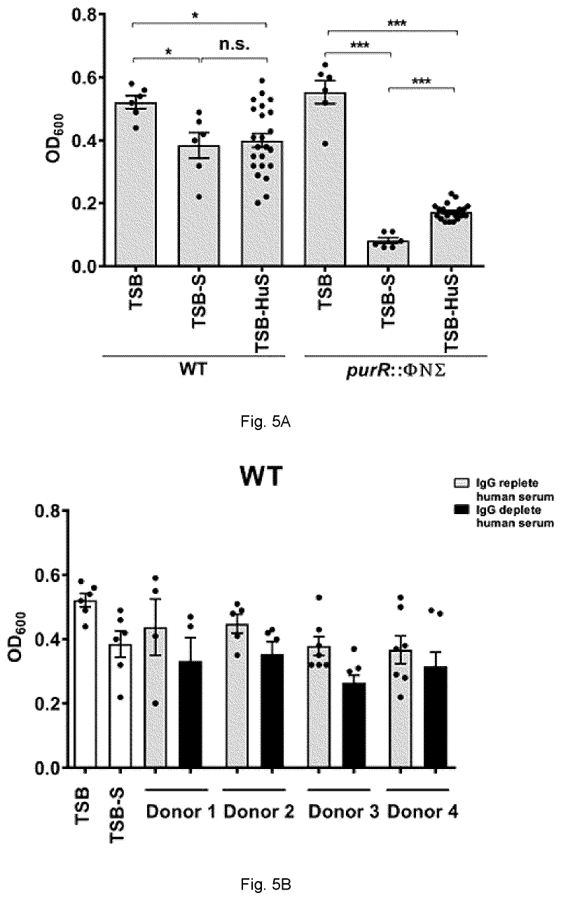

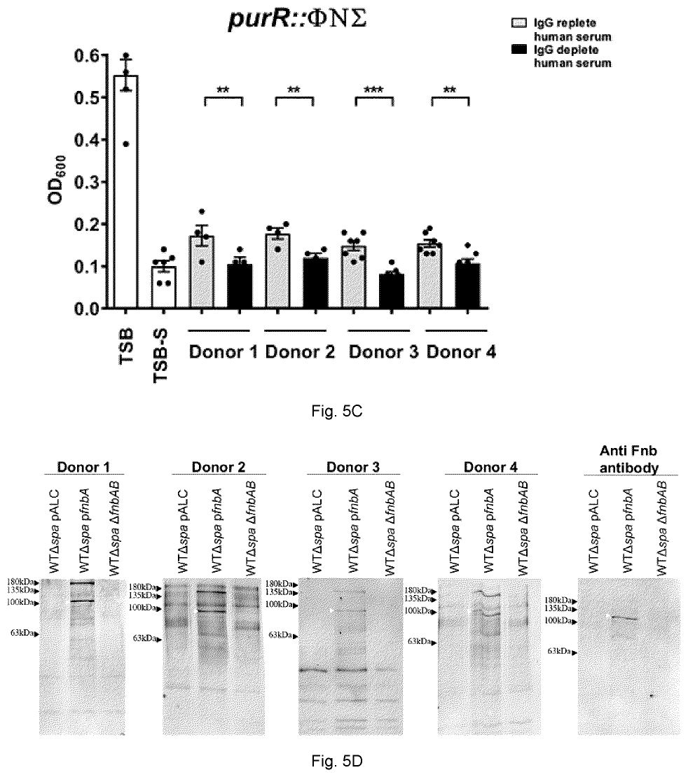

Thus far the present data have shown that purR mutations can be selected for under stress, that purR inactivation leads to exaggerated clumping in the presence of HS and that purR mutants of S. aureus are hypervirulent in a systemic murine model of infection. Despite this, human infection by S. aureus occurs with high frequency and yet the striking consequences of purR deletion have not been noted in humans. A search of publicly available whole genome sequences identified a non-synonymous change or changes in the purR gene in 331 of 8201 sequences (see Table 5). However, few details on the infection type or outcome were available and, at this point, no correlations could be drawn between the presence of a purR mutation and disease severity. To begin to explore this further in the laboratory setting, we next tested whether human serum can support hyper-clumping of purR::ΦNΣ bacteria. We isolated fresh human serum from healthy volunteers and this serum was used to assay for clumping as described above. When the WT and purR::ΦNΣ mutant were grown in TSB with 10% v/v human serum (TSB-HuS), the purR::ΦNΣ clumped, when compared to the WT, but the clumping observed was less pronounced than that seen in horse serum (FIG. 5A). Since the clumping phenotype relies on FnbA and FnbB, and their interaction with Fn, we hypothesised that anti-FnBP (i.e. blocking) antibodies are present in human serum, since humans are exposed to S. aureus throughout their lifetime, and that these antibodies would interfere with clumping. To test this, we passaged human serum over a protein A column, thus removing most of the IgG. TSB containing 10% v/v IgG-depleted human serum showed increased levels of clumping for the purR::ΦNΣ mutant, when compared to TSB-HuS, but no significant difference was observed for the WT (FIGS. 5B and 5C). Moreover, addition of the purified human IgG to the purR::ΦNΣ mutant growing in TSB-S resulted in significant amelioration of the clumping phenotype (FIG. 12). This indicates that antibodies present in human serum can interfere with the purR-dependent clumping phenotype. To determine whether some of these immunoglobulins are indeed anti-FnbA/B antibodies we utilized Western blot analysis to test for the ability of human serum to detect FnbA/B protein. No signal could be detected for the WT, likely due to low expression, which is in agreement with our luminescence findings (FIG. 3A), prompting our use of an fnbA overexpression construct in the WT background instead. In agreement with our assertion, we were indeed able to demonstrate reactivity to S. aureus proteins, including FnbA/B, with human serum (FIG. 5D), corroborating our hyper-clumping data. These data indicate that humans can carry anti-FnbA/B antibodies that, while not necessarily protective against S. aureus infection, may confer protection against the clumping-dependent hyper-virulent purR phenotype.

Anti-FnBP Antibodies Protect Against purR Hypervirulence

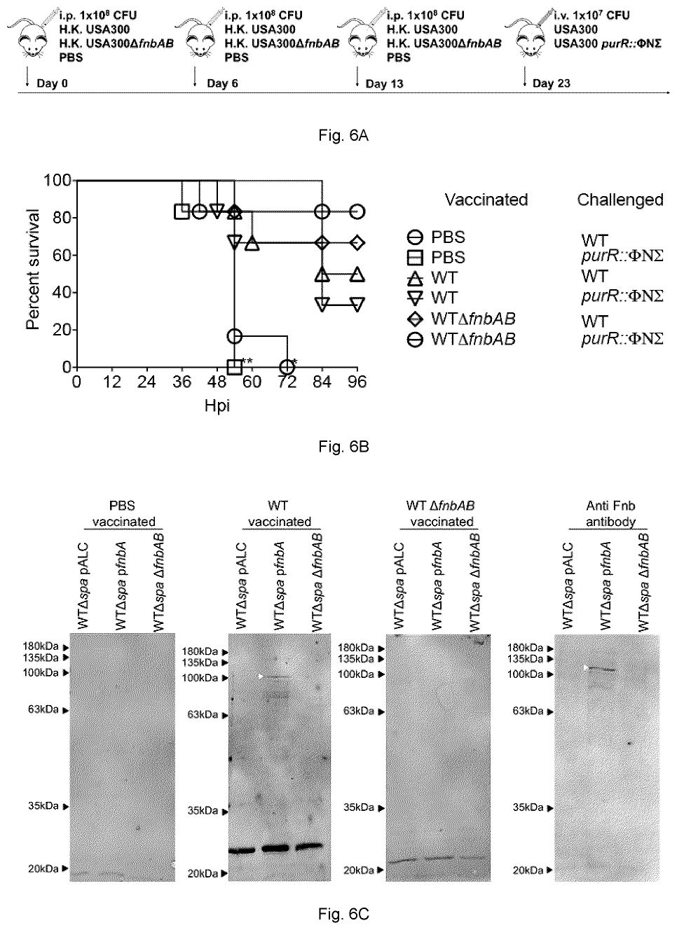

Given our data indicated that anti-FnbA/B antibodies present in human serum can impair purR mutant clumping, we hypothesised that mice with antibodies recognizing the FnBPs would be protected from hypervirulence associated with purR::ΦNΣ infection. To test this, we vaccinated groups of 12 mice intraperitoneally with either 1×10⁸ heat-killed (HK) USA300, 1×10⁸ HK USA300ΔfnbAB, or with PBS on day 0, 6 and 13 (FIG. 6A). On day 23, animals in each group were challenged with either live WT USA300 or USA300 purR::ΦNΣ bacteria. In groups vaccinated with WT USA300, significantly more animals survived challenge with the purR::ΦNΣ strain, when compared to those vaccinated with USA300ΔfnbAB or the vehicle control (FIG. 6B). Serum from vaccinated animals demonstrated that mice receiving HK WT USA300 raised antibodies towards S. aureus antigens, including FnbA/B, while those challenged with HK USA300ΔfnbAB likewise raised antibodies to many antigens, but not to FnbA/B proteins (FIG. 6C), indicating the protective response is indeed due to anti-FnbA/B antibodies.

Altogether, these data indicate that overexpression of purR reduces, even eliminates the formation of S. aureus lesions. Antibodies against S. aureus FnbA/B can protect against the hypervirulent phenotype associated with the loss of purR function, a mutation that we have demonstrated can arise during infection.

An agent that increases the number of wild-type purine biosynthesis repressor (purR) protein in bacteria, or an interfering agent that inhibits, competes, or titrates binding of a fibronectin binding protein in the bacteria to fibronectin, such as an anti-FnbA/B antibody, can be used to attenuate, prevent or treat an infection or disorder caused by or associated with bacteria in subjects (human or other animals). For example, the agent or interfering agent can be used as prophylactic (proactive) in high risk situations, such as when a subject is about to undergo surgery, undergoing dialysis, or in immunocompromised subjects (such as very young subjects, old subjects, sick subjects, cancer patient undergoing immune suppression, and so forth) to anticipate, forestall and/or preclude infections altogether. Subjects that may even have pre-existing anti-FnbA/B antibodies will also benefit from an agent that increases the number of wild-type purine biosynthesis repressor (purR) protein in the bacteria, or from an interfering agent that inhibits, competes, or titrates binding of a fibronectin binding protein in the bacteria to fibronectin, such as an anti-FnbA/B antibody, to increase the subject's defenses and prevent or anticipate infections altogether.

TABLE 1
Bacterial strains used in this study

| Strain | Description | Source |
|---|---|---|
| S. aureus | | |
| USA300 LAC | CA-MRSA; cured of resistance plasmids | Lab stock |
| RN4220 | r_{K}⁻ m_{K}⁺; capable of accepting foreign | Lab stock |
| | DNA | |
| SH1000 | WT S. aureus strain derived from 8325-4 | Lab stock |
| SH1000 | Strain SH1000 containing a transposon | This study |
| purR::ΦNΣ | insertion in the purR gene | |
| SH1000 | Strain SH1000 containing a transposon | This study |
| purR::ΦNΣ + | insertion in the purR gene and a plasmid | |
| ppurR | carrying a full-length purR gene | |
| RN6390 | WT S. aureus strain | Lab stock |
| RN6390 | Strain RN6390 containing a transposon | This study |
| purR::ΦNΣ | insertion in the purR gene | |
| RN6390 | Strain RN6390 containing a transposon | This study |
| purR::ΦNΣ + | insertion in the purR gene and a plasmid | |
| ppurR | carrying a full-length purR gene | |
| MN8 | WT S. aureus strain | Lab stock |
| MN8 | Strain MN8 containing a transposon | This study |
| purR::ΦNΣ | insertion in the purR gene | |
| MN8 | Strain MN8 containing a transposon | This study |
| purR::ΦNΣ + | insertion in the purR gene and a plasmid | |
| ppurR | carrying a full-length purR gene | |
| Newman | WT S. aureus strain | Lab stock |
| Newman | Strain Newman containing a transposon | This study |
| purR::ΦNΣ | insertion in the purR gene | |
| Newman | Strain Newman containing a transposon | This study |
| purR::ΦNΣ + | insertion in the purR gene and a plasmid | |
| ppurR | carrying a full-length purR gene | |
| USA300 | Strain USA300 LAC containing a | This study |
| purR::ΦNΣ | transposon insertion in the purR gene | |
| USA300 | Strain USA300 LAC containing a | This study |
| purR::ΦNΣ + | transposon insertion in the purR gene | |
| ppurR | and a plasmid carrying a full-length | |
| | purR gene | |
| USA300 ΔfnbAB | Strain USA300 LAC with a complete | This study |
| | deletion of the fnbA and fnbB genes | |
| USA300 | Strain USA300 LAC with a complete | This study |
| purR::ΦNΣ | deletion of the fnbA and fnbB genes | |
| ΔfnbAB | and a transposon insertion in the purR | |
| | gene | |
| USA300 ΔspaΔsbi | Strain USA300 with a complete deletion | This study |
| | in the spa and sbi genes | |
| USA300 ΔspaΔsbi | Strain USA300 LAC with a complete | This study |
| ΔfnbAB | deletion of the fnbA and fnbB genes and | |
| | the spa and sbi genes | |
| USA300 ΔspaΔsbi | Strain USA300 with a complete deletion | This study |
| purR::ΦNΣ | in the spa and sbi genes and a transposon | |
| | insertion in the purR gene | |
| USA300 ΔspaΔsbi | Strain USA300 LAC with a complete | This study |
| ΔfnbAB | deletion of the fnbA and fnbB genes and | |
| purR::ΦNΣ | the spa and sbi genes and a transposon | |
| | insertion in the purR gene | |
| USA300 purR^{R96A} | Strain USA300 LAC with a R96A SNP | This study |
| | in the purR gene | |
| USA300 purR^{Q52P} | Strain USA300 LAC with a Q52P SNP | This study |
| | in the purR gene | |
| USA300 | Strain USA300 LAC with a Q240Stop | This study |
| purR^{Q240Stop} | SNP in the purR gene | |
| USA300 | Strain USA300 LAC with a S172Stop | This study |
| purR^{S172Stop} | SNP in the purR gene | |
| USA300 | Strain USA300 LAC with a V156E SNP | This study |
| purR^{V156E} | in the purR gene | |
| USA300 | Strain USA300 LAC with a V229 | This study |
| purR^{V229frameshift} | frameshift SNP in the purR gene | |
| E. coli | | |
| DH5α | FΦ80IacZΔM15Δ(lacZYAargF)u169 | Promega |
| | recA1 endA 1 hsdR17 (rK,mK⁺) phoA | |
| | supE44λthi1 gyrA96 relA 1 | |

TABLE 2
Primers used in this study.

| SEQ | | |
|---|---|---|
| ID | | |
| NO | Primer name | Primer sequence |
| 17 | PurR F | TTTGGTACCATATCTTGAAAAGTGGTGCAGAT |
| | | GG |
| 18 | PurR R | TTTGAGCTCCCTGCTTCTTCCAAAACAACCTT |
| | | TA |
| 19 | pALC MCS F | ATACCGCACAGATGCGTAAGG |
| 20 | pALC MCS R | CGATGACTTAGTAAAGCACATCTAA |
| 21 | FnbAB Up F | GGGGACAAGTTTGTACAAAAAAGCAGGCTCAC |
| | | AGATACTTCCAAGATTCTCAAACC |
| 22 | FnbAB Up R | GGACCTCCGCGGCAGTGGAACAAGGTAAAGTA |
| | | GTAACAC |
| 23 | FnbABDown F | GGACCTCCGCGGGTATTCAAGTCATCAGAAAC |
| | | CCTTGTC |
| 24 | FnbABDown R | GGGGACCACTTTGTACAAGAAAGCTGGGTCAG |
| | | GGCCTATATTTAACAAAGTTGCAC |
| 25 | pGYluxFnbA F | GCGCCCGGGGCAATATATTGCCTTGAAACACG |
| 26 | pGYluxFnbA R | GCGGTCGACTATAATATCTCCCTTTAAATGC |
| 27 | pGYluxFnbB F | GCGCCCGGGGTGTTTTCTGATTGCTTCATTGC |
| 28 | pGYluxFnbB R | GCGGTCGACTATAATATTCTCCCTTAAATGC |
| 29 | PurR His F | TTTGGATCCGGTCCAAGTGCTTCCGGTAA |
| 30 | PurR His R | TTTCATATGAGATATAAACGAAGCGAGAGA |
| 31 | PurE F | GGGCAGTTCTTCCGATTGGA |
| 32 | PurE R | CTGTTCGCCCTTGACTGCTA |
| 33 | FnbA F | TTTGGATCCTGTGCGTATTGTACAGGCGA |
| 34 | FnbA R | TTTGAGCTCAGCCGTATTTCAAGCCGACA |
| 35 | qPCR PurE F | CTTCTGAAGCGAGAGAAAGAGGTATAA |
| 36 | qPCR PurE R | CAATAACTGGTAGCGTCGTTAATGATG |
| 37 | qPCR FnbA F | CGGCATTAGAAAACATAAATTGGG |
| 38 | qPCR FnbA R | GTTTTATTATCAGTAGCTGAATTCCC |
| 39 | qPCR FnbB F | GAAAACACAAATTGGGAGCG |
| 40 | qPCR FnbB R | TGTTTCGCTTGCTTTACTTTC |

TABLE 3
Gene expression changes in purR::ΦNΣ mutant,
as measured by RNAseq

| | Log2 Fold | |
|---|---|---|
| Gene (Locus tag) | change | P value |
| PurN (SAUSA300_0974) | 4.2 | 0 |
| PurH (SAUSA300_0975) | 4.17 | 5.88E−13 |
| PurQ (SAUSA300_0970) | 4.13 | 0 |
| PurC (SAUSA300_0968) | 4.12 | 0 |
| PurS (SAUSA300_0969) | 4.04 | 0 |
| PurM (SAUSA300_0973) | 4.02 | 1.11E−16 |
| PurL (SAUSA300_0971) | 4 | 3.67E−12 |
| PurD (SAUSA300_0976) | 3.87 | 9.69E−14 |
| PurK (SAUSA300_0967) | 3.77 | 5.55E−16 |
| PurF (SAUSA300_0972) | 3.75 | 1.67E−15 |
| PurE ((SAUSA300_0966) | 3.64 | 2.55E−15 |
| tRNA-Asn | 2.63 | 0.08 |
| tRNA-Ala | 2.44 | 0.0003 |
| tRNA-Ala | 2.19 | 0.001 |
| purB (SAUSA300_1889) | 2.06 | 0.0001 |
| purA (SAUSA300_0017) | 1.85 | 2.37E−05 |
| FnbA (SAUSA300_2441) | 0.24 | 0.65 |
| FnbB (SAUSA300_2440) | −1.81 | 0.001 |
| clfA (SAUSA300_0772) | 0.31 | 0.45 |
| clfB (SAUSA300_2565) | −0.13 | 0.78 |
| Xpt (SAUSA300_0386) | −0.69 | 0.4 |
| icaA (SAUSA300_260.0) | −4.71 | 0.0007 |

TABLE 4
Quantification of lesion frequency 24 hpi

| | | | | |
|---|---|---|---|---|
| Heart | Kidney | Liver | Spleen | Lung |

Frequency of lesions

| | | | | | |
|---|---|---|---|---|---|
| WT pALC | 1 | 1 | 1 | 1 | 1 |
| WT pALC | 1 | 1 | 1 | 1 | 1 |
| purR::ΦNΣ pALC | 2 | 3 | 1 | 1 | 1 |
| purR::ΦNΣ pALC | 2 | 3 | 0 | 1 | 1 |
| purR::ΦNΣ ppurR | 0 | 0 | 0 | 0 | 0 |
| purR::ΦNΣ ppurR | 0 | 0 | 0 | 0 | 0 |

Severity of lesions

| | | | | | |
|---|---|---|---|---|---|
| WT pALC | 1 | 2 | 1 | 1 | 1 |
| WT pALC | 1 | 2 | 1 | 1 | 1 |
| purR::ΦNΣ pALC | 2.5 | 3 | 1 | 1 | 1 |
| purR::ΦNΣ pALC | 2.5 | 3 | 0 | 1 | 1 |
| purR::ΦNΣ ppurR | 0 | 0 | 0 | 0 | 0 |
| purR::ΦNΣ ppurR | 0 | 0 | 0 | 0 | 0 |

Frequency x severity

| | | | | | |
|---|---|---|---|---|---|
| WT pALC | 1 | 2 | 1 | 1 | 1 |
| WT pALC | 1 | 2 | 1 | 1 | 1 |
| purR::ΦNΣ pALC | 5 | 9 | 1 | 1 | 1 |
| purR::ΦNΣ pALC | 5 | 9 | 0 | 1 | 1 |
| purR::ΦNΣ ppurR | 0 | 0 | 0 | 0 | 0 |
| purR::ΦNΣ ppurR | 0 | 0 | 0 | 0 | 0 |

TABLE 5
(Accession number followed by mutation type)

1190472077, L115I; 1190473252, L115I; 1263964561, K35 STOP; 1235891243, L232F;
1235890392, L232F; 1235872284, L232F; 1235860490, L232F; 1041151790, L232F;
861944504, G267S; 875894747, I122V; 875894524, I122V; 875898312, I122V; 875920513,
I122V; 875899845, I122V; 875900059, I122V; 875900354, I122V; 875900856, I122V;
960331061, V201I; 587194894, E92D; 587196163, E92D; 875900648, I122V; 581608047,
P266H; 579937160, L114I; 997826329, L274S; 579698444, F33I; 997712924, L274S;
995867920, L274S; 814566315, V201I; 814566368, V201I; 814566240, V201I; 1029559408,
F262L; 814566266, V201I; 1029547851, F262L; 997769248, N268S; 997560967, V229I;
926126488, T70M; 857881076, D245H; 857860252, D245H; 857876982, D245H; 910631648,
V201I; 910683539, V201I; 857852602, D245H; 857849258, D245H; 910735437, V201I;
910451404, V201I; 910602936, V201I; 912469779, V201I; 910570687, V201I; 910046839,
V201I; 910509629, V201I; 910372277, V201I; 910414352, V201I; 910396341, V201I;
910377950, V201I; 910046048, V201I; 910046619, V201I; 910367246, V201I; 910046531,
V201I; 910046436, V201I; 910046365, V201I; 910046300, V201I; 910046213, V201I;
910371319, V201I; 910370088, V201I; 910046149, V201I; 910363026, V201I; 910045871,
V201I; 910044219, V201I; 910045794, V201I; 910043635, V201I; 910040091, L232F;
910045705, V201I; 910045494, V201I; 910045407, V201I; 910045327, V201I; 910045257,
V201I; 910045184, V201I; 910045141, V201I; 910044609, V201I; 910044459, V201I;
910044386, V201I; 910044295, V201I; 910044154, V201I; 910044066, V201I; 910043987,
V201I; 910043933, V201I; 910043772, V201I; 910043041, V201I; 910040879, V201I;
910040729, V201I; 910039867, V201I; 910039790, V201I; 910039643, V201I; 910039303,
V201I; V201I; 910038196, V201I; 910037245, V201I; 910037755, V201I; 910037090, V201I;
910037536, V201I; 910037653, V201I; 910037845, V201I; 910037425, V201I; 910036493,
V201I; 910036577, V201I; 910037017, V201I; 910036926, V201I; 910036848, V201I;
910036759, V201I; 910036658, V201I; 910036390, V201I; 910027656, N268S; 910026922,
V229I; 667528927, V201I; 667529132, V201I; 319438722, V201I; 570296852, Q34 STOP;
477747068, F33I; 723153210, M12I; 421957249, V201I; 414081978, M12I; 1069074423, L56
STOP; 579059981, L121 STOP; 925215635, K34 STOP; 910038913, K34 STOP; 584236157,
K34 STOP; 618800447, MULTIPLE; 1275296202, V229I; 1069077617, V30STOP;
1069077617, I40 STOP; 1042772831 V229I; 1042772276, V229I; 1015560674, H225Y;
1015560724, H225Y; 1025627356, V229I; 1025626860, V229I; 1237442475, truncated at I24;
1184257856, H225Y; 580365707, T58I; 1072466828, V229I; 1072544971, V229I; 1072459302,
V229I; 1072682328, V229I; 1072669974, V229I; 1072695792, V229I; 1072666391, V229I;
1072687463, V229I; 1072487502, V229I; 1072613579, V229I; 1072674258, V229I;
1072663938, V229I; 1072679559, V229I; 1072691428, V229I; 1072626566, V229I;
1072634535, V229I; 1072479884, V229I; 1072658488, V229I; 1072615920, V229I;
1072650393, V229I; 1072623924, V229I; 1029304020, E78V; 394329061, V229I; 570297808,
S41 STOP; 1175582508, K34 STOP; 1190478114, R2 STOP; 1218205675, S197L; 930070352,
R2 STOP; 930070253, R2 STOP; 930070269, R2 STOP; 930070165, R2 STOP; 930070121, R2
STOP; 930070076, A224V; 930070017, A224V; 930069994, A224V; 1270591801, K37T;
1270595092, K37T; 1270584928, K37T; 1270580755, K37T; 1270571043, K37T; 1270564114,
K37T; 1270570220, K37T; 1270586607, K37T; 1270587568, K37T; 1270579636, K37T;
875932980, S177L; 930070063, Q240R; 653579470, R8I; 875927145, S177L; 1272401461,
K37T; 600573462, V83I; 600511395, V83I; 593115873, N196K; 581788901, V83I; 581425047,
V83I; 581412653, V83I; 581368019, V83I; 581311741, V83I; 581236793, V83I; 581230248,
V83I; 580100621, A208G; 580028546, A208G; 579964606, A208G; 579956826, A208G;
579901852, A208G; 579847672, A208G; 579716164, A208G; 579665542, A208G; 579641094,
A208G; 579629371, A208G; 579597576, A208G; 579571734, V83I; 579554582, A208G;
579537640, A208G; 579380251, A208G; 579378586, A208G; 579361785, A208G;
1143531124, R2 STOP; 1143531056, R2 STOP; 1143531630, V202I; 1143530907, R2 STOP;
1143531184, R2 STOP; 1143531261, R2 STOP; 1143530984, R2 STOP; 1143530888, R2
STOP; 1029861413, V148I; 1072557970, A224V; 1072408331, R2 STOP; 1072584484,
A224V; 1072410915, R2 STOP; 1029630097, V83I; 1029706154, A138T; 664805869, Q52R;
664805250, Q52R; 997256084, L56I; 664805431, Q52R; 477945486, V83I; 477854412, V83I;
478125304, V83I; 478104946, V83I; 341848884, A224V; 927328118, V30 STOP; 875940378,
E7 STOP; 1105664827, Y126F; 375022900, V30 STOP; 932894922, S41 STOP; 600507407,
V10A; 1024329861, S274H; 582759289, P25T; 1072500503, 140L S41I; 580911623, V30
STOP; 1029201620, N116D; 593741899, T89A; 827326431, MULTIPLE; 1190494570, R8
STOP; 600573681, MULTIPLE; 1237729931, I9 STOP; 1181852756, L242 TRUNCATED;
1145794992, L242 TRUNCATED; 875925813, S113T E128D; 600283536, S113T E128D;
599761857, S113T E128D; 1109731787, S113T E128D; 1109734354, S113T E128D;
1109729280, S113T E128D; 1109741623, S113T E128D; 1109724289, S113T E128D;
1105940614, S113T E128D; 1105977300, S113T E128D; 1105919770, S113T E128D;
1105787320, S113T E128D; 1105779564, S113T E128D; 1106069630, S113T E128D;
857608414, S113T E128D; 857605639, S113T E128D; 1109749458, S113T E128D N263K ;
1105579255, S113T E128D; 678254705, S113T E128D; 1105710487, S113T E128D;
678257374, S113T E128D N263K; 678247281, S113T E128D N263K; 678252281, S113T
E128D N263S; 678259863, S113T E128D N263S; 678262528, S113T E128D N263S;
678265158, S113T E128D N263S; 678270357, S113T E128D N263S; 678273346, S113T
E128D N263S; 678249824, Y175 TRUNCATED; 678267660, K55Q E78A L90H E92Q
S113T K130Q I151V K251R N263K; 1072736005, K55Q E78A L90H E92Q S113T
K130Q I151V K251R N263K; 875909596, K55Q E78A L90H E92Q S113T K130Q
I151V K251R N263K; 875927088, K55Q E78A L90H E92Q S113T K130Q I151V
K251R N263K; 875933566, K55Q E78A L90H E92Q S113T K130Q I151V K251R
N263K; 875937156, K55Q E78A L90H E92Q S113T K130Q I151V K251R N263K;
875940329, multiple; 875940454, multiple; 875946555, K55Q E78A L90H E92Q S113T
K130Q I151V K251R N263K; 645287611, multiple; 875939496, multiple; 827313769,
multiple; 1125656615, multiple; 1070264237, L26 STOP; 861932800, M1 STOP; 1237627715,
K4 STOP; 1072730652, M1 STOP; 1237723438, E7 STOP; 1072728488, MULTIPLE;
874346830, MULTIPLE; 613107659, MULTIPLE; 1237618233, R2 STOP; 1070261762,
MULTIPLE; 582930284, F51 TRUNCATED; 910485516, K4 STOP; 1181848461, Y3 STOP;
910714303, MULTIPLE; 910587863, M1 STOP; 910651850, MULTIPLE; 875940357,
MULTIPLE; 910651336, K3 STOP; 910679582, MULTIPLE; 910701638, R2 STOP;
910648504, STOP; 910646999, STOP; 910574494, STOP; 910572911, STOP; 910570841,
STOP; 910378073, STOP; 897320957, STOP; 910687983, STOP; 910570881, STOP;
910570881, STOP; 910378523, STOP; 910639176, STOP.

TABLE 6
B. subtilis WWWHVCGAAYRWTW (SEQ ID NO: 41)
L. lactis AWWWCCGAACWWT (SEQ ID NO: 42)
purE - 130 tcaaaataaagttcgatttttgattgaaaaagcaga
aattgcttgttatgctatatctataatatacaac -
60 (SEQ ID NO: 43)
purA - 130 aaaacgatttgttaaaatgatttttcttttaaaaag
gccgaaaatcaatgttcgatttttatttgcatta -
60 (SEQ ID NO: 44)
fnbA - 130 aaaattaatgacaatcttaacttttcattaactcgc
ttttttgtattgcttttaaaaaccgaacaatata -
60 (SEQ ID NO: 45)

Sequence Listing

>Wild-Type PurR Protein Sequence (SEQ ID NO: 1)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGVSVLVESKEVKQRLIEDYTSLVKLSDVDEYNQEFNVEPGNSLSKFS
>PurR Q52P (SEQ ID NO: 2)
MRYKRSERIVFMTQYLMNHPNKLIPLTFFVKKFKQAKSSISEDV
QIIKNTFPKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGVSVLVESKEVKQRLIEDYTSLVKLSDVDEYNQEFNVEPGNSLSKFS
>Deletion affecting V229 → frame shift (SEQ ID NO: 3)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGstop
>Y71stop (SEQ ID NO: 4)
MRYKRSERIVFMTQYLMNHPNKLIPLTFFVKKFKQAKSSISEDV
QIIKNTFQKEKLGTVITTAGASGGVTstop
>V156E (SEQ ID NO: 5)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPEVVIR
KDNKVTEGSTVSINYVSGSSRKIETMVLSKRTLAENSNVLVVDDFMRAGGSINGVMNL
MNEFKAHVKGVSVLVESKEVKQRLIEDYTSLVKLSDVDEYNQEFNVEPGNSLSKFS
>S172stop (SEQ ID NO: 6)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVstop
>H225D (SEQ ID NO: 7)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKADVKGVSVLVESKEVKQRLIEDYTSLVKLSDVDEYNQEFNVEPGNSLSKFS
>S230top (SEQ ID NO: 8)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGVstop
>Q240stop (SEQ ID NO: 9)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGVSVLVESKEVKstop
>R96A (SEQ ID NO: 10)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKEALLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAENSNVLVVDDFMRAGGSINGVMNL
MNEFKAHVKGVSVLVESKEVKQRLIEDYTSLVKLSDVDEYNQEFNVEPGNSLSKFS
>V148F (SEQ ID NO: 11)
MRYKRSERIVFMTQYLMNHPNKLIPLTFFVKKFKQAKSSISEDV
QIIKNTFQKEKLGTVITTAGASGGVTYKPMMSKEEATEVVNEVITLLEEKERLLPGGY
LFLSDLVGNPSLLNKVGKLIASIYMEEKLDAVVTIATKGISLANAFANILNLPVVVIR
KDNKVTEGSTVSINYVSGSSRKIETMVLSKRTLAENSNVLVVDDFMRAGGSINGVMNL
MNEFKAHVKGVSVLVESKEVKQRLIEDYTSLVKLSDVDEYNQEFNVEPGNSLSKFS
>Q45stop (SEQ ID NO: 12)
MRYKRSERIVFMTQYLMNHPNKLIPLTFFVKKFKQAKSSISEDVstop
>Q14stop (SEQ ID NO: 13)
MRYKRSERIVFMTstop
>Insertion affecting L91 → frame shift (SEQ ID NO: 14)
MRYKRSERIVFMTQYLMNHPNKLIPLTFFVKKFKQAKSSISEDV
QIIKNTFQKEKLGTVITTAGASGGVTYKPMMSKEEATEVVNEVITLstop
>Deletion affecting N196 → frame shift (SEQ ID NO: 15)
MRYKRSERIVFMTQYLMNHPNKLIPLTFFVKKFKQAKSSISEDV
QIIKNTFQKEKLGTVITTAGASGGVTYKPMIVISKEEATEVVNEVITLLEEKERLLPGGY
LFLSDLVGNPSLLNKVGKLIASIYMEEKLDAVVTIATKGISLANAVANILNLPVVVIR
KDNKVTEGSTVSINYVSGSSRKIETMVLSKRTLAEstop

### REFERENCES

1. Gorwitz, R. J. J. et al. Changes in the prevalence of nasal colonization with Staphylococcus aureus in the United States, 2001-2004. J. Infect. Dis. 197, 1226-1234 (2008).
2. Wertheim, H. F. L. et al. The role of nasal carriage in Staphylococcus aureus infections. Lancet Infect. Dis. 5, 751-762 (2005).
3. Tong, S. Y. C., Davis, J. S., Eichenberger, E., Holland, T. L. & Fowler, V. G. Staphylococcus aureus infections: epidemiology, pathophysiology, clinical aanifestations, and management. Clin. Microbiol. Rev. 28, 603-661 (2015).
4. Klevens, R., MA, M., Nadle, J. & al, et. Invasive methicillin-resistant Staphylococcus aureus infections in the united states. JAMA 298, 1763-1771 (2007).
5. Foster, T. J. The remarkably multifunctional fibronectin binding proteins of Staphylococcus aureus. Eur. J. Clin. Microbiol. Infect. Dis. 35, 1923-1931 (2016).
6. Geoghegan, J. A. & Foster, T. J. Cell wall-anchored surface proteins of Staphylococcus aureus: many proteins, multiple functions. in Staphylococcus aureus: Microbiology, Pathology, Immunology, Therapy and Prophylaxis (eds. Bagnoli, F., Rappuoli, R. & Grandi, G.) 95-120 (Springer International Publishing, 2017). doi: 10.1007/82_2015_5002
7. Alonzo, F. & Torres, V. J. The bicomponent pore-forming leucocidins of Staphylococcus aureus. Microbiol. Mol. Biol. Rev. 78, 199-230 (2014).
8. Cheung, G. Y. C., Joo, H.-S., Chatterjee, S. S. & Otto, M. Phenol-soluble modulins—critical determinants of staphylococcal virulence. FEMS Microbiol. Rev. 38, 698-719 (2014).
9. Seilie, E. S. & Wardenburg, J. B. Staphylococcus aureus pore-forming toxins: the interface of pathogen and host complexity. Semin. Cell Dev. Biol. 72, 101-116 (2017).
10. Koymans, K. J., Vrieling, M., Gorham, R. D. & van Strijp, J. A. G. Staphylococcal immune evasion proteins: structure, function, and host adaptation. in Staphylococcus aureus: Microbiology, Pathology, Immunology, Therapy and Prophylaxis (eds. Bagnoli, F., Rappuoli, R. & Grandi, G.) 441-489 (Springer International Publishing, 2017). doi:10.1007/82_2015_5017
11. Rooijakkers, S. H. M., van Kessel, K. P. M. & van Strijp, J. A. G. Staphylococcal innate immune evasion. Trends Microbiol. 13, 596-601 (2005).
12. Le, K. Y. & Otto, M. Quorum-sensing regulation in staphylococci—an overview. Frontiers in Microbiology 6, 1174 (2015).
13. Liu, Q., Yeo, W. & Bae, T. The SaeRS two-component system of Staphylococcus aureus. Genes (Basel). 7, (2016).
14. Somerville, G. A. & Proctor, R. A. At the crossroads of bacterial metabolism and virulence factor synthesis in Staphylococci. Microbiol. Mol. Biol. Rev. 73, 233-248 (2009).
15. Harper, L. et al. Staphylococcus aureus responds to the central metabolite pyruvate to regulate virulence. MBio 9, (2018).
16. Sun, F. et al. Aureusimines in Staphylococcus aureus are not involved in virulence. PLoS One 5, 1-11 (2011).
17. Flack, C. E. et al. Differential regulation of staphylococcal virulence by the sensor kinase SaeS in response to neutrophil-derived stimuli. Proc. Natl. Acad. Sci. 111, E2037 LP-E2045 (2014).
18. Harraghy, N. et al. sae is essential for expression of the staphylococcal adhesins Eap and Emp. Microbiology 151, 1789-1800 (2005).
19. Baroja, M. L. et al. The SaeRS two-component system is a direct and dominant transcriptional activator of toxic shock syndrome toxin 1 in Staphylococcus aureus. J. Bacteriol. 198, 2732 LP-2742 (2016).
20. Xiong, Y. Q., Willard, J., Yeaman, M. R., Cheung, A. L. & Bayer, A. S. Regulation of Staphylococcus aureus α-toxin gene (hla) expression by agr, sarA and sae in vitro and in experimental infective endocarditis. J. Infect. Dis. 194, 1267-1275 (2006).
21. Fey, P. D. et al. A Genetic resource for rapid and comprehensive phenotype screening of nonessential Staphylococcus aureus genes. MBio 4, (2013).
22. Lipinski, B., Hawiger, J. & Jeljaszewicz, J. Staphylococcal clumping with soluble fibrin monomer complexes. J. Exp. Med. 126, 979 LP-988 (1967).
23. Pietrocola, G. et al. Molecular interactions of human plasminogen with fibronectin-binding protein B (FnBPB), a fibrinogen/fibronectin-binding protein from Staphylococcus aureus. J. Biol. Chem. 291, 18148-18162 (2016).
24. Grundmeier, M. et al. Truncation of fibronectin-binding proteins in Staphylococcus aureus strain Newman leads to deficient adherence and host cell invasion due to loss of the cell wall anchor function. Infect. Immun. 72, 7155-7163 (2004).
25. Herman-Bausier, P., El-Kirat-Chatel, S., Foster, T. J., Geoghegan, J. A. & Dufrêne, Y. F. Staphylococcus aureus Fibronectin-Binding Protein A Mediates Cell-Cell Adhesion through Low-Affinity Homophilic Bonds. MBio 6, e00413-15 (2015).
26. Jendresen, C. B., Martinussen, J. & Kilstrup, M. The PurR regulon in Lactococcus lactis—transcriptional regulation of the purine nucleotide metabolism and translational machinery. Microbiology 158, 2026-2038 (2012).
27. Bera, A. K., Zhu, J., Zalkin, H. & Smith, J. L. Functional Dissection of the Bacillus subtilis pur Operator Site. J. Bacteriol. 185, 4099-4109 (2003).
28. Weng, M., Nagy, P. L. & Zalkin, H. Identification of the Bacillus subtilis pur operon repressor. Proc. Natl. Acad. Sci. U.S.A 92, 7455-7459 (1995).
29. He, B., Smith, J. M. & Zalkin, H. Escherichia coli purB gene: cloning, nucleotide sequence, and regulation by purR. J. Bacteriol. 174, 130-136 (1992).
30. Hamdallah, I. et al. Experimental Evolution of Escherichia coli K-12 at High pH and RpoS Induction. Appl. Environ. Microbiol. 84, e00520-18 (2018).
31. Li, L. et al. Role of Purine Biosynthesis in Persistent Methicillin-Resistant Staphylococcus aureus (MRSA) Infection. J. Infect. Dis. (2018). doi:10.1093/infdis/jiy340
32. Wermser, C. & Lopez, D. Identification of Staphylococcus aureus genes involved in the formation of structured macrocolonies. Microbiology 164, 801-815 (2018).
33. Yee, R., Cui, P., Shi, W., Feng, J. & Zhang, Y. Genetic Screen Reveals the Role of Purine Metabolism in Staphylococcus aureus Persistence to Rifampicin. Antibiotics 4, 627-642 (2015).
34. Mei, J. M., Nourbakhsh, F., Ford, C. W. & Holden, D. W. Identification of Staphylococcus aureus virulence genes in a murine model of bacteraemia using signature-tagged mutagenesis. Mol. Microbiol. 26, 399-407 (1997).
35. Connolly, J. et al. Identification of Staphylococcus aureus factors required for pathogenicity and growth in human blood. Infect. Immun. 85, (2017).
36. Benton, B. M. et al. Large-scale identification of genes required for full virulence of Staphylococcus aureus. J Bacteriol 186, 8478-8489 (2004).
37. Gratani, F. L. et al. Regulation of the opposing (p)ppGpp synthetase and hydrolase activities in a bifunctional RelA/SpoT homologue from Staphylococcus aureus. PLOS Genet. 14, e1007514 (2018).
38. Geiger, T., Kastle, B., Gratani, F. L., Goerke, C. & Wolz, C. Two small (p)ppGpp synthases in Staphylococcus aureus mediate tolerance against cell envelope stress conditions. J. Bacteriol. 196, 894-902 (2014).
39. Reiβ, S. et al. Global analysis of the Staphylococcus aureus response to mupirocin. Antimicrob. Agents Chemother. 56, 787-804 (2012).
40. Edwards, A. M., Potts, J. R., Josefsson, E. & Massey, R. C. Staphylococcus aureus host cell invasion and virulence in sepsis is facilitated by the multiple repeats within FnBPA. PLoS Pathog. 6, (2010).
41. Que, Y.-A. et al. Fibrinogen and fibronectin binding cooperate for valve infection and invasion in Staphylococcus aureus experimental endocarditis. J. Exp. Med. 201, 1627-1635 (2005).
42. Shinji, H. et al. Role of fibronectin-binding proteins A and B in in vitro cellular infections and in vivo septic infections by Staphylococcus aureus. Infect. Immun. 79, 2215-2223 (2011).
43. Wolz, C. et al. Agr-independent regulation of fibronectin-binding protein(s) by the regulatory locus sar in Staphylococcus aureus. Mol. Microbiol. 36, 230-243 (2000).
44. Saravia-Otten, P., Müller, H. P. H.-P. & Arvidson, S. Transcription of Staphylococcus aureus fibronectin binding protein genes is negatively regulated by agr and an agr-independent mechanism. J. Bacteriol. 179, 5259-5263 (1997).
45. Heilmann, C. et al. Staphylococcus aureus_fibronectin-binding protein

(FnBP)-mediated adherence to platelets, and aggregation of platelets induced by FnBPA but not by FnBPB. J. Infect. Dis. 190, 321-329 (2004).
46. Fitzgerald, J. R. et al. Fibronectin-binding proteins of Staphylococcus aureus mediate activation of human platelets via fibrinogen and fibronectin bridges to integrin GPIIb/IIIa and IgG binding to the FcγRIIa receptor. Mol. Microbiol. 59, 212-230 (2006).
47. Etz, H. et al. Identification of in vivo expressed vaccine candidate antigens from Staphylococcus aureus. Proc. Natl. Acad. Sci. 99, 6573 LP-6578 (2002).
48. Casolini, F. et al. Antibody response to fibronectin-binding adhesin FnbpA in patients with Staphylococcus aureus infections. Infect. Immun. 66, 5433 LP-5442 (1998).
49. DeJonge, M. et al. Clinical trial of safety and efficacy of INH-A21 for the prevention of nosocomial staphylococcal bloodstream infection in premature infants. J. Pediatr. 151, 260-5, 265.e1 (2007).
50. Benjamin, D. K. et al. A blinded, randomized, multicenter study of an intravenous Staphylococcus aureus immune globulin. J. Perinatol. 26, 290-295 (2006).
51. Rupp, M. E. et al. Phase II, randomized, multicenter, double-blind, placebo-controlled trial of a polyclonal anti-Staphylococcus aureus capsular polysaccharide immune globulin in treatment of Staphylococcus aureus bacteremia. Antimicrob. Agents Chemother. 51, 4249-4254 (2007).
52. Shinefield, H., Black, S., Fattom, A., Horwith, G. & al, E. Use of Staphylococcus aureus conjugate vaccine in patients receiving hemodialysis. N. Engl. J. Med. 346, 491-496 (2002).
53. Bae, T. & Schneewind, O. Allelic replacement in Staphylococcus aureus with inducible counter-selection. Plasmid 55, 58-63 (2006).
54. Luong, T. T. & Lee, C. Y. Improved single-copy integration vectors for Staphylococcus aureus. J. Microbiol. Methods 70, 186-190 (2007).
55. Huesca, M. et al. Synthetic peptide immunogens elicit polyclonal and monoclonal antibodies specific for linear epitopes in the D motifs of Staphylococcus aureus fibronectin-binding protein, which are composed of amino acids that are essential for fibronectin binding. Infect. Immun. 68, 1156-1163 (2000).
56. Schaeffer, C. R. et al. Accumulation-associated protein enhances Staphylococcus epidermidis biofilm formation under dynamic conditions and as required for infection in a rat catheter model. Infect. Immun. 83, 214-226 (2015).
57. Foster T J. 2019. Surface Proteins of Staphylococcus aureus. Microbiol Spectr 7:1-22.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method of attenuating, preventing or treating an infection or disorder in a subject caused by or associated with bacteria, comprising administering to the subject (a) an agent that increases the number of wild-type purine biosynthesis repressor (purR) protein in the bacteria, or (b) an interfering agent that that inhibits, competes, or titrates binding of a fibronectin binding protein in the bacteria to fibronectin.

2. The method of claim 1, wherein the interfering agent that inhibits, competes, or titrates binding of the fibronectin binding protein in the bacteria to fibronectin comprises an antibody or antigen binding fragment that specifically recognizes or binds the fibronectin binding protein.

3. The method of claim 1, wherein the agent that increases the number of wild-type PurR protein in the bacteria comprises one or more of:
a phage carrying copies of a wild-type purR gene;
a conjugative plasmid that can conjugate with the bacterium carrying copies of the wild-type purR gene;
a non-naturally occurring Clustered Regularly Interspaced Short Palindromic Repeats (CRISPR)-CRISPR associated (Cas) system comprising (i) a first regulatory element operable in the bacteria operably linked to at least one nucleotide sequence encoding a CRISPR-Cas system guide RNA that hybridizes with a target DNA sequence in a DNA molecule of the bacteria, and (ii) a second regulatory element operable in the bacteria operably linked to a nucleotide sequence encoding a Cas9 protein, wherein components (i) and (ii) are located on same or different vectors of the system, whereby the guide RNA targets the target DNA sequence and the Cas9 protein cleaves the DNA molecule, and thereby resulting in overxpression of the wild type purR gene in the bacteria; and, wherein the Cas9 protein and the guide RNA do not naturally occur together; or
wild-type purR protein or a fragment thereof conjugated to a carrier that transfers the wild-type conjugated purR protein or fragment thereof to the bacteria having the mutated purR gene.

4. The method of claim 3, wherein the carrier is a liposome, a micelle, or a pharmaceutically acceptable polymer.

5. The method of claim 1, wherein the bacteria carry a purR gene or a biological equivalent of the purR gene.

6. The method of claim 1, wherein the bacteria carry a mutant purR gene.

7. The method of claim 1, wherein the bacteria are E. coli, S. aureus, or Bacillus subtilis.

8. The method of claim 1, wherein the bacteria are S. aureus.

9-27. (canceled)

28. A recombinant bacterium that expresses a polypeptide encoded by a mutant purR gene.

29. The recombinant bacterium of claim 28, wherein the polypeptide is any of SEQ ID NO.: 2 to SEQ ID NO.:15.

30-32. (canceled)

33. A mutant purine biosynthesis repressor (purR) polypeptide that confers hypervirulent phenotype in a bacterium.

34. The purR mutant polypeptide of claim 33, wherein the purR polypeptide comprises an amino acid sequence according to any one of SEQ ID Nos. 2 to 15.

35. (canceled)

36. A nucleic acid that encodes the purR polypeptide of claim 34.

37. A polypeptide that is at least 70% identical to the purR polypeptide of claim 34, and exhibits substantially equivalent biological activity to the purR polypeptide of claim 34.

38. A polypeptide that is encoded by a polynucleotide that hybridizes under stringent conditions to a complement of the nucleic acid of claim 36, and exhibits substantially equivalent biological activity to the polypeptide encoded by said nucleic acid.
